(19) United States
(12) Patent Application Publication
(10) Pub. No.: US 2026/0086079 A1
(43) Pub. Date: Mar. 26, 2026

(54) FUNCTIONALIZED GRAPHENE AND METHODS OF USE THEREOF

(71) Applicants: Matthew Hummer, SHAKER HEIGHTS, OH (US); Gregory J. Hummer, SHAKER HEIGHTS, OH (US)

(72) Inventors: Matthew Hummer, SHAKER HEIGHTS, OH (US); Gregory J. Hummer, SHAKER HEIGHTS, OH (US)

(21) Appl. No.: 19/403,970

(22) Filed: Nov. 30, 2025

Related U.S. Application Data
(63) Continuation-in-part of application No. 19/260,530, filed on Jul. 6, 2025.
Application No. 19/260,530 is a continuation of application No. 18/888,132, filed on Sep. 17, 2024, now Pat. No. 12,517,108.

(51) Int. Cl. G01N 33/487; G01N 33/569; G16H 10/40

(52) U.S. Cl. CPC G01N 33/48792; G01N 33/48714; G01N 33/4875; G01N 33/56983; G16H 10/40

(57) ABSTRACT

A graphene-based composition is disclosed comprising functionalized graphene powder produced by detonation, plasma synthesis, liquid phase exfoliation, mechanical exfoliation among other graphene production methods known in the art. The graphene powder includes oxygen-containing functional groups such as hydroxyl, epoxy, carbonyl, and carboxyl, with a carbon-to-oxygen atomic ratio ranging from approximately 1.0 to 99.0. The powder is dispersed in an organic solvent, non-organic solvent, aqueous medium, surfactant, or POSS-based medium at a solids concentration between about 2% w/v and 30% w/v. Methods of functionalizing the graphene powder and forming dispersions are provided, along with methods of making ink dispersions with polymer binders and printed or coated articles formed therefrom. The graphene formulations exhibit tunable surface chemistry, controlled defect structures as measured by Raman spectroscopy, and stable dispersion properties suitable for applications in printed electronics, conductive coatings, sensing platforms, and lightweight composites.

### CROSS-REFERENCE TO RELATED APPLICATIONS

This patent application is a Continuation-in-part and claims priority to the United States Patent Application entitled: “FUNCTIONALIZED GRAPHENE AND METHODS OF USE THEREOF”, U.S. Ser. No. 19/260,530 filed on Jul. 6, 2025 by Matthew Hummer, which is a Continuation of United States patent application entitled: “SIMULTANEOUS DISEASE DETECTION SYSTEM METHOD AND DEVICES”, U.S. Ser. No. 18/888,132 filed on Sep. 17, 2024 by Matthew Hummer, which is a Continuation of United States patent application entitled: “DEVICE FOR READING, PROCESSING AND TRANSMITTING TEST RESULT DATA FOR PATHOGENS OR VIRUSES IN FLUID TEST SAMPLES”, U.S. Ser. No. 18/896,643 filed on Sep. 25, 2024 by Matthew Hummer, which is a Continuation-in-part of United States patent application entitled: “GRAPHENE INK COMPOSITION AND METHODS OF USE THEREOF”, U.S. Ser. No. 18/928,124 filed on Oct. 27, 2024 by Matthew Hummer, which is a Continuation-in-part of United States patent application entitled: “DEVICE FOR READING, PROCESSING AND TRANSMITTING TEST RESULT DATA FOR PATHOGENS OR VIRUSES IN FLUID TEST SAMPLES”, U.S. Ser. No. 18/896,643 filed on Sep. 25, 2024 by Matthew Hummer, which is a Continuation Continuation-in-part of United States patent application entitled: “DEVICE FOR READING, PROCESSING AND TRANSMITTING TEST RESULT DATA FOR PATHOGENS OR VIRUSES IN FLUID TEST SAMPLES”, U.S. Ser. No. 18/440,925 filed on Feb. 13, 2024 by Matthew Hummer, which is a Continuation-in-part of United States patent application entitled: “DEVICE FOR READING, PROCESSING AND TRANSMITTING TEST RESULT DATA FOR PATHOGENS OR VIRUSES IN FLUID TEST SAMPLES”, U.S. Ser. No. 17/505,611 filed on Oct. 19, 2021 by Matthew Hummer, which is a Continuation-in-part of United States patent application entitled: “METHOD AND DEVICES FOR DETECTING VIRUSES AND BACTERIAL PATHOGENS”, U.S. Ser. No. 17/324,085 filed on May 18, 2021 by Matthew Hummer, which is a Continuation-in-part of United States patent application entitled: “METHOD AND DEVICES FOR DETECTING VIRUSES AND BACTERIAL PATHOGENS”, U.S. Ser. No. 16/926,701 filed on Jul. 11, 2020 by Gregory J. Hummer, which is a Continuation Continuation-in-part of United States patent application entitled: “METHOD AND DEVICES FOR DETECTING CHEMICAL COMPOSITIONS AND BIOLOGICAL PATHOGENS”, U.S. Ser. No. 16/926,702 filed on Jul. 11, 2020 by Gregory J. Hummer, which is a Continuation-in-part of United States patent application entitled: “MONITORING SYSTEM FOR USE WITH MOBILE COMMUNICATION DEVICE”, U.S. Ser. No. 16/513,753 filed on Jul. 17, 2019 by Gregory J. Hummer, which is a Continuation-in-part of United States patent application entitled: “MONITORING SYSTEM FOR USE WITH MOBILE COMMUNICATION DEVICE”, U.S. Ser. No. 15/891,410 filed on Feb. 8, 2018 by Gregory J. Hummer, Patented, which is a Continuation in-part of United States patent application entitled: “MONITORING SYSTEM FOR USE WITH MOBILE COMMUNICATION DEVICE”, U.S. Ser. No. 15/235,981 filed on Aug. 12, 2016 by Gregory J. Hummer, which claims benefit of provisional United States Provisional Patent Application U.S. Ser. No. 62/297,385 filed on Feb. 19, 2016, all the U.S. Patent Applications being incorporated herein by reference in their entirety.

### BACKGROUND OF THE INVENTION

Functionalized graphene has emerged as a critical material for printed electronics, coatings, and advanced composites due to its high conductivity, tunable surface chemistry, and mechanical strength. However, scalable production of graphene powders with controlled oxygen-containing functional groups, dispersibility in multiple solvent systems, and reproducible structural properties remains a challenge.

Existing graphene production methods often lack the ability to simultaneously control the degree of oxidation, structural disorder, and dispersion stability. Moreover, conventional functionalization techniques frequently disrupt the graphene lattice, resulting in diminished electronic performance or inconsistent flake morphology. Additionally, there is limited understanding of how electrokinetic properties such as zeta potential relate to graphene's surface area, crystallinity, and defect state, which are essential for ink formulation and device reliability.

There remains a need for a composition and method that enables the production of functionalized graphene powder with tunable carbon-to-oxygen atomic ratios, controlled Raman and surface properties, and dispersion stability across aqueous and organic systems. The present invention addresses these challenges by providing a system and method for producing, characterizing, and dispersing functionalized graphene powder optimized for printability, substrate adhesion, and electrical performance.

### SUMMARY OF THE INVENTION

The present invention provides compositions, methods and articles based on functionalized graphene powder characterized by controlled oxygen functionalization, dispersibility, and structural uniformity.

In one embodiment, the invention provides a composition comprising functionalized graphene powder dispersed in at least one solvent system and embedded within at least one polymer matrix. The composition may further include additives such as dispersing agents, wetting agents/surface tension modifiers, rheology modifiers/thickeners, binders/film formers, adhesion promoters, crosslinkers/curing agents, humectants/solvent retention aids, antifoaming agents, and pH buffers. The graphene powder includes oxygen-containing functional groups such as hydroxyl, epoxy, ether, carbonyl, carboxyl and ester, and is defined by a carbon-to-oxygen atomic ratio ranging from about 1.0 to about 99.0. The powder is dispersed in solvents selected from water, organic, inorganic, surfactant-based, or POSS-based systems at a solids concentration between about 2% and 30% w/v. The composition may further include polymers such as polyurethane (PU), polyacrylate/acrylics, epoxy, polyvinyl alcohol (PVA), polyvinylpyrrolidone (PVP), polyethylene glycol (PEG), polyimide precursors and cellulose derivatives (e.g. hydroxypropyl cellulose (HPC), carboxymethyl cellulose (CMC), hydroxyethyl cellulose (HEC) and ethyl cellulose) for forming printable or sprayable conductive composites.

The invention also provides methods for producing such functionalized graphene powder using exfoliation and activation techniques including plasma synthesis, detonation, sonication, milling, chemical vapor deposition (CVD), exfoliation, cavitation and other production methods. Functionalization may involve plasma oxidation, wet chemistry, POSS grafting, or silanization. Dispersion properties, crystallinity, and defect structure are monitored using Raman spectroscopy, zeta potential analysis and other characterization techniques included in this disclosure.

In certain embodiments, zeta potential is shown to correlate with specific surface area, crystallinity, flake orientation, and Raman band ratios, enabling predictive control over printability, dispersion stability, conductivity and substrate adhesion. The invention further provides dispersions suitable for various deposition methods and printed articles retaining conductivity and surface functionality post-deposition.

### BRIEF DESCRIPTION OF THE DRAWINGS

FIG. 1 shows, for illustrative purposes only, an example of graphene ink components of one embodiment.

FIG. 2 shows a block diagram of an overview flow chart of oxidized graphene ink production of one embodiment.

FIG. 3 shows a block diagram of an overview of oxidized graphene ink production of one embodiment.

FIG. 4 shows a block diagram of an overview of graphene ink with 2-5% w/v solids content of one embodiment.

FIG. 5 shows a block diagram of an overview of graphene ink with 5-10% w/v solids content of one embodiment.

FIG. 6 shows a block diagram of an overview of graphene ink with 10-25% w/v solids content of one embodiment.

FIG. 7 shows a block diagram of an overview of graphene ink with 25-50% w/v solids content of one embodiment.

FIG. 8 shows a block diagram of a dual-film sensor of one embodiment.

### SUMMARY OF THE INVENTION

The embodiments disclose a graphene ink composition including a thermal plasma device configured to produce an oxidized graphene. The composition comprises oxidized graphene, a hydrophobic fluid component, and a cellulose derivative. Wherein the hydrophobic fluid component comprises at least one from a group of alcohols, glycols, terpenes, terpene alcohols, or combinations thereof, and wherein the composition comprises a predetermined graphene-cellulose derivative solids concentration based on the method of depositing the material. A dispensed oxidized graphene ink is dispensed onto a substrate in a predetermined pattern. The substrate may be a base for circuit boards found in applications in fields including electronics, energy storage, sensors, biomedical devices, and biotechnology devices. The pattern may be produced with various deposition techniques (e.g., inkjet, aerosol, screen, pad, or flexographic printing, spin coating, spray coating, chemical vapor deposition, physical vapor deposition, epitaxial growth, etc.), and viscosity, adhesion, and other characteristics of the oxidized graphene ink can play a pivotal role in the quality of the printing process.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized, and structural changes may be made without departing from the scope of the present invention.

It should be noted that the descriptions that follow, for example, in terms of ink compositions and methods for preparing the ink compositions and depositing or transferring, or printing the ink compositions on substrates, are described for illustrative purposes, and the underlying composition and methods for preparing compositions can apply to any number of applications, including biosensing applications.

In one embodiment, the apparatus for biosensing applications comprises a biosensing platform for detecting predetermined analytical targets of a sample, the biosensing platform comprising: a conductive sensor having a plurality of printed electrodes on the conductive sensor, wherein the plurality of electrodes is configured to be functionalized with depositing sequence-specific single-stranded DNA (ssDNA) probes, a plurality of biosensors coupled to the plurality of printed electrodes and the predetermined analytical targets and having a measurement device configured to record measurements of changes in electrical current of current passing through the plurality of printed electrodes when in contact with the predetermined analytical targets, at least one digital analytical device coupled to the plurality of biosensors and configured to use the measurements to identify an existence and a type of the predetermined analytical targets in the sample, and at least one communication device coupled to the digital analytical device and configured to transmit the existence and the type of predetermined analytical targets in the sample to a remote device.

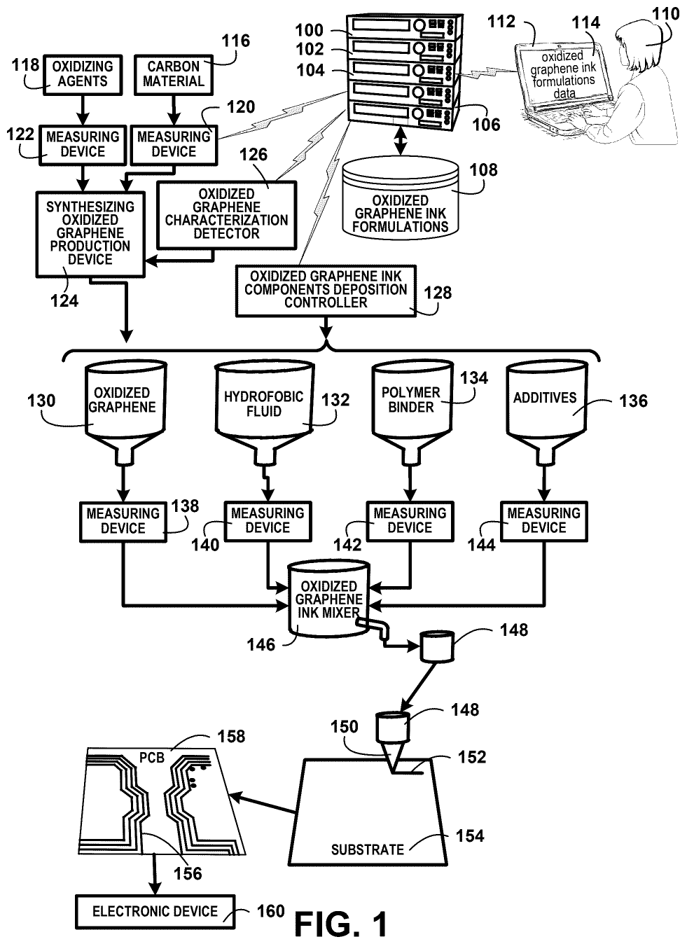

FIG. 1 shows, for illustrative purposes only, an example of graphene ink components of one embodiment. FIG. 1 shows an overview of a manufacturing process to produce oxidized graphene inks. The oxidized graphene inks are used in deposition techniques, primarily printing by various methods for various electronic devices, and conductive boards for various applications. The various printing techniques (e.g., inkjet, aerosol, screen, pad, or flexographic printing, spin coating, spray coating, etc.) use different viscosities, adhesion characteristics, and flow rates, to name but a few. The applications of printed circuit boards are installed in numerous types of devices in fields including electronics, energy storage, sensors, biomedical devices, and biotechnology devices, to name a few. Because the devices conduct differing voltages and currents, the oxidized graphene ink used has characteristics that are suited to the use conditions. Hence, there are specific compositions of materials and chemicals to produce effective oxidized graphene inks to fit the application use. The formulations of the specific compositions in this instance are recorded and selectable by the user to meet the characteristics for order specifications. FIG. 1 shows an overview of the manufacturing process and control system to produce the oxidized graphene ink to meet the characterized in the order specification.

FIG. 1 shows a remote server 100 having a plurality of communication devices 102, a plurality of processors 104, and artificial intelligence and machine learning 106. An oxidized graphene ink formulations database 108 coupled to the remote server 100 is used to display oxidized graphene ink formulations on a remote computer 112. The remote computer 112, coupled to the remote server 100 using a user graphical interface, displays selectable oxidized graphene ink formulations data 114 for a user 110. The user 110 determines which formulation to select to produce a specific oxidized graphene ink product. The user 110 selects the specific formulation to produce an oxidized graphene ink for a specific use. The remote computer 112, coupled to the remote server 100, shows the formulation characteristics display 114 to allow the user 110 to make an informed determination.

The selected formulation wirelessly transmits predetermined quantities of carbon material 116 and oxidizing agents 118 that are measured using a first measuring device 120 for the carbon material 116 and a separate second measuring device 122 for measuring the oxidizing agents 118. The measured quantities are deposited into a reactor vessel. A synthesizing oxidized graphene production device 124 uses multiple mixing and stirring devices to produce a uniform reaction. The mixture is analyzed by an oxidized graphene characterization detector 126 to determine the size of the oxidized graphene. The artificial intelligence and machine learning 106 compare the size of the oxidized graphene to the order specification and previous productions of the same formulation to confirm consistency.

An oxidized graphene ink components deposition controller 128 using the predetermined quantities of the oxidized graphene ink components to produce the oxidized graphene ink based on the specific formulation. Predetermined quantities of oxidized graphene 130, hydrophobic fluid 132, polymer binder 134, and additives 136 are measured using separate measuring devices 138-144 and deposited into an oxidized graphene ink mixer 146. The oxidized graphene ink mixture is deposited into an oxidized graphene ink dispensing supply container 148. The oxidized graphene ink dispensing supply container 148 is configured in multiple styles to attach to different oxidized graphene ink dispensing devices 150 to supply the oxidized graphene ink mixture to the specific dispensing device. A dispensed oxidized graphene ink 152 is shown being dispensed onto a substrate 154 in a predetermined pattern. A completed oxidized graphene ink printed circuit 156 is created for a printed circuit board (PCB) or other circuit configuration. In this example, the PCB 158 is installed in an electronic device 160 or other type of device using printed circuits of one embodiment.

FIG. 1 shows an overview of graphene ink components comprising van der Waal materials, including oxidized graphene, hydrophobic fluid, polymer binder, and ink additives. The graphene ink composition of the present invention is prepared by dispersing oxidized graphene in a hydrophobic fluid component, including alcohols, terpenes, nonpolar solvents, organic solvents, hydrocarbons, silicone oils, fluorinated fluids, chlorinated fluids, surfactants, and blends of hydrophobic fluid components.

The production of oxidized graphene includes processes, for example, plasma-produced, reduced graphene oxide, graphene oxide, holey graphene, single-layer graphene, multi-layer graphene, functionalized graphene, chemical vapor deposition, and physical vapor deposition, which introduce oxygen-containing functional groups onto the graphene surface. These functional groups improve the graphene's compatibility with polar solvents and facilitate its dispersion in ink formulations. Additionally, the particle size of oxidized graphene is determined to increase ink stability and printing performance, with smaller particle sizes generally leading to improved dispersion and film quality.

Van der Waal materials include graphene, arranged in a two-dimensional honeycomb lattice having a single layer of carbon atoms, which have properties, including high conductivity, mechanical strength, and flexibility. As a result, van der Waal materials, including graphene, have found applications in fields such as electronics, energy storage, sensors, biomedical devices, and biotechnology devices. Graphene inks allow for the deposition of graphene-based materials onto diverse substrates through multiple deposition and transfer techniques. The ink depositions offer scalability, cost-effectiveness, and versatility in device fabrication. However, the successful formulation of graphene inks poses several challenges. Achieving proper dispersion and stabilization of graphene flakes in a liquid medium is critical to ensure uniform printing and high-performance devices. Moreover, controlling the rheological properties of graphene inks, including viscosity, surface tension, particle size, zeta potential, density, and other rheological characteristics, is essential for precise deposition, transfer, and patterning of van der Waal materials, including graphene.

Mixing the oxidized graphene in a hydrophobic fluid with at least one polymer binder, including cellulose derivatives, polyvinylpyrrolidone (PVP), polyvinyl alcohol (PVA), polyethylene glycol (PEG), polyurethane (PU), polystyrene (PS), acrylic polymers, epoxy resins, and polyimides, is dispersed in the hydrophobic fluid. In at least one embodiment, ink additives include defoaming components, dispersing components, wetting components, adhesion components, conductivity enhancers, rheology modifiers, cross-linking agents, plasticizers, surfactants, cross linking agents, UV stabilizers, flame retardants, and a combination thereof. The resulting composition exhibits excellent deposition capability, film forming, and adhesion to various substrates, making it suitable for a wide range of applications.

The adhesion-promoting component, including silicone, silane, urea, amino-functional, phosphate esters, organtitanates, polyolefin-based compounds, maleic anhydride, polyurethane, epoxy resins, acrylic-based compounds, polyvinyl butyral, ethylene vinyl acetate copolymer, primers, blocked isocyanate compounds, or a combination thereof, improves the adhesion of the graphene ink to substrates. Improved adhesion of the graphene ink to substrates, improves the performance and reliability of printed devices. The anti-foaming component, comprising organic polymers, copolymers, silicon-based polymers, polymeric compounds, hydrophobic particles, solvents, surfactants, carriers, or a combination thereof, prevents foam formation during ink preparation and printing processes, ensuring consistent print quality.

In one embodiment, the incorporation of polymers, particularly cellulose derivatives, polyvinylpyrrolidone (PVP), polyvinyl alcohol (PVA), polyethylene glycol (PEG), polyurethane (PU), polystyrene (PS), acrylic polymers, epoxy resins, polyimides, and a combination thereof into graphene inks stabilizes the formulation of the ink. For example, cellulose derivatives are biocompatible and film-forming polymers that act as a binder and stabilizer for graphene flakes, improving their dispersion and adhesion to substrates. Additionally, the inclusion of hydrophobic fluid components and additives further improves the ink's rheological properties and compatibility with methods of deposition.

The oxidized graphene used in the composition has an oxygen content of at least 4 atomic percent (at %) as measured by X-ray photoelectron spectroscopy (XPS), with a particle size ranging from 100 to 3,000 nm. In at least one preferred method of production includes using plasma-enhanced chemical vapor deposition or thermal plasma methods, where carbon-containing gases and hydrogen-containing gases, including methane, ethylene, acetylene, hydrogen, argon, and oxygen, are used as raw materials. It should be appreciated that other methods include mechanical exfoliation, chemical vapor deposition, physical vapor deposition, liquid phase exfoliation, epitaxial growth on silicon carbide, reduction of graphene oxide, unzipping carbon nanotubes, molecular assembly, and electronic exfoliation, among other methods of synthesizing and producing graphene.

Polymer binders, including cellulose derivatives, are used with various hydrophobic fluid solvents to form stable graphene ink formulations. Additionally, the use of hydrophobic fluid components, including alcohols, alkanes, and terpenes, can aid in dispersing graphene and improving film formation.

There exists a use for graphene ink compositions that offer tunable properties, including solids concentration, viscosity, and compatibility with deposition methods. Such compositions would enable the facile deposition or transfer of graphene-based materials onto substrates, opening avenues for the development of advanced electronic devices, energy storage, sensors, and other applications.

The present invention creates novel ink compositions using van der Waal materials, including graphene comprising oxidized graphene, hydrophobic fluid components, polymer binders, and additives. These compositions offer customizable properties tailored to specific deposition, transfer, and printing requirements, facilitating the fabrication of graphene-based devices with improved performance and functionality.

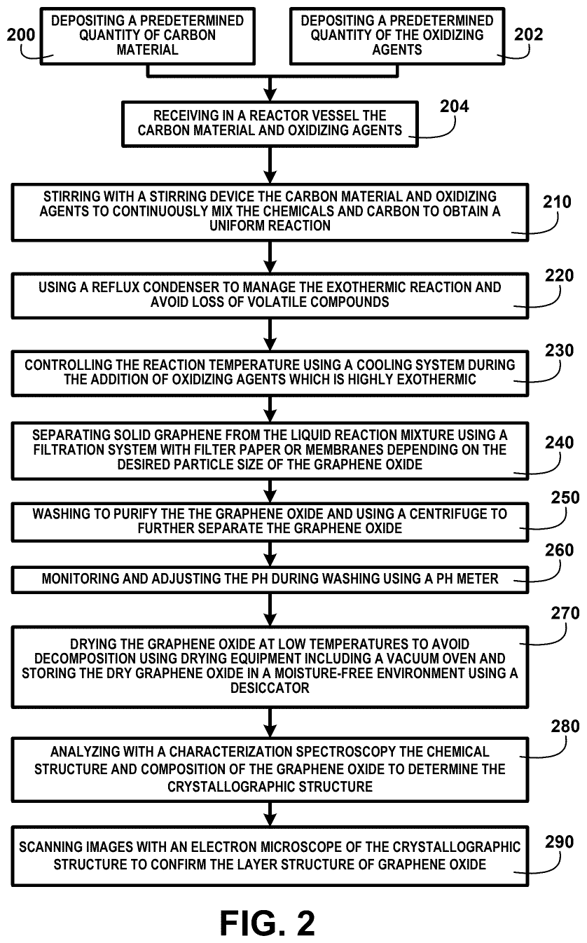

FIG. 2 shows a block diagram of an overview flow chart of oxidized graphene ink production of one embodiment. FIG. 2 shows depositing a predetermined quantity of the carbon material 200 and depositing a predetermined quantity of the oxidizing agents 202. Receiving in a reactor vessel the deposited carbon material and oxidizing agents 204. Stirring with a stirring device the carbon material and oxidizing agents to continuously mix the chemicals and carbon to obtain a uniform reaction 210. Using a reflux condenser to manage the exothermic reaction and avoid loss of volatile compounds 220 in the depositions. Controlling the reaction temperature using a cooling system during the addition of oxidizing agents, which are highly exothermic 230. Separating solid graphene from the liquid reaction mixture using a filtration system with filter paper or membranes, depending on the desired particle size of the graphene oxide 240. Washing to purify the graphene oxide and using a centrifuge to further separate the graphene oxide 250. Monitoring the pH during washing using a pH meter 260 wherein the pH meter reading activates a pH-adjustment subsystem configured to introduce an acid, base, or buffer to achieve a predetermined target pH level.

Drying the graphene oxide at low temperatures to avoid decomposition using drying equipment, including a vacuum oven, and storing the dry graphene oxide in a moisture-free environment using a desiccator 270. Analyzing with a characterization spectroscopy, the chemical structure and composition of the graphene oxide to determine the crystallographic structure 280. Scanning images with an electron microscope of the crystallographic structure to confirm the layer structure of graphene oxide 290. The oxidizing of the carbon material prepares the graphene oxide as a component of the oxidized graphene ink of one embodiment.

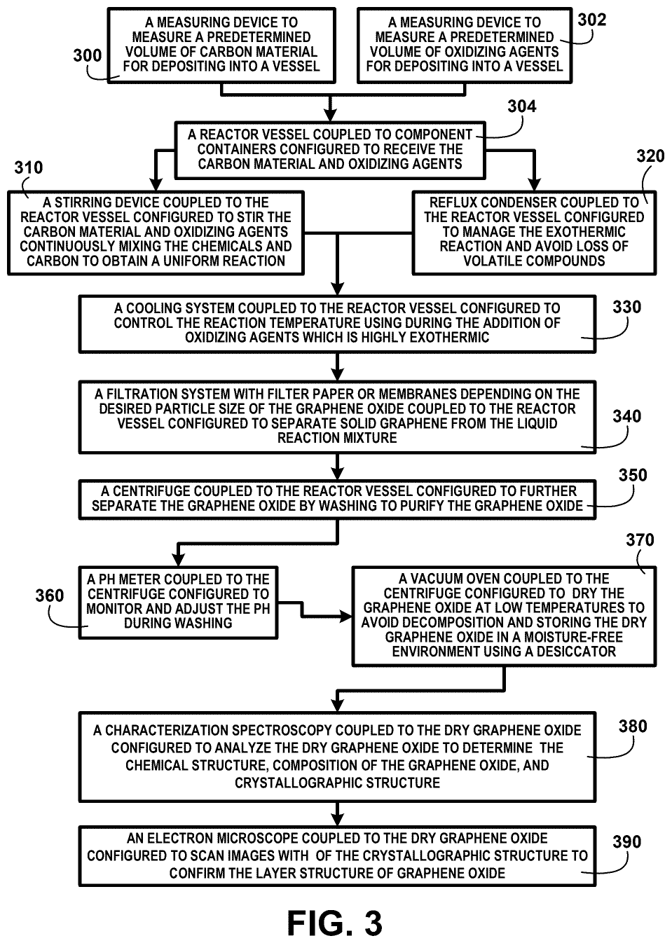

FIG. 3 shows a block diagram of an overview of oxidized graphene ink production of one embodiment. FIG. 3 shows a measuring device to measure a predetermined volume of carbon material for depositing into a vessel 300 and a measuring device to measure a predetermined volume of oxidizing agents for depositing into a vessel 302. A reactor vessel coupled to component containers configured to receive the carbon material and oxidizing agents 304. A stirring device coupled to the reactor vessel is configured to stir the carbon material and oxidizing agents, continuously mixing the chemicals and carbon to obtain a uniform reaction 310. A reflux condenser coupled to the reactor vessel is configured to manage the exothermic reaction and avoid loss of volatile compounds 320. A cooling system coupled to the reactor vessel is configured to control the reaction temperature used during the addition of oxidizing agents, which are highly exothermic 330. A filtration system with filter paper or membranes, depending on the desired particle size of the graphene oxide, coupled to the reactor vessel, configured to separate solid graphene from the liquid reaction mixture 340. A centrifuge coupled to the reactor vessel is configured to further separate the graphene oxide by washing to purify the graphene oxide 350. A pH meter coupled to the centrifuge is configured to monitor and adjust the pH during washing 360. A vacuum oven coupled to the centrifuge is configured to dry the graphene oxide at low temperatures to avoid decomposition, and storing the dry graphene oxide in a moisture-free environment using a desiccator 370. A characterization spectroscopy coupled to the dry graphene oxide is configured to analyze the dry graphene oxide to determine the chemical structure, composition of the graphene oxide, and crystallographic structure 380. An electron microscope coupled to the dry graphene oxide is configured to scan images of the crystallographic structure to confirm the layer structure of graphene oxide 390 of one embodiment.

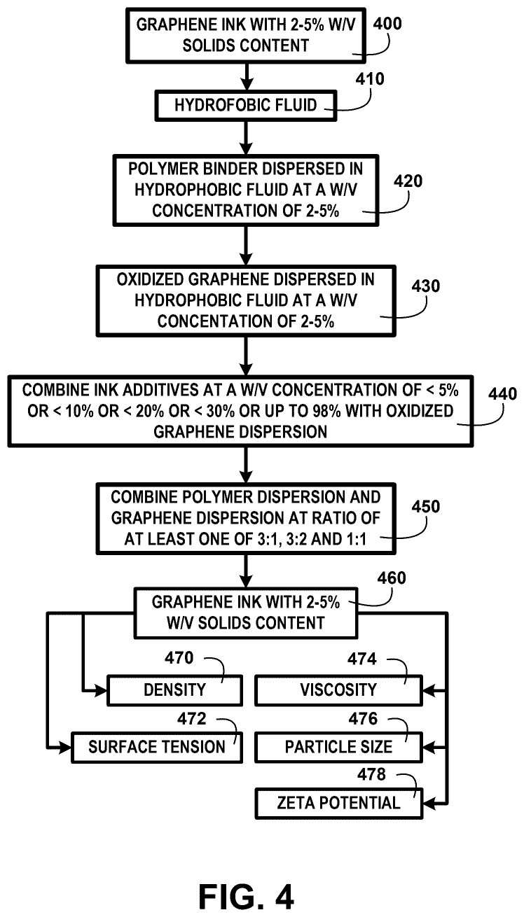

FIG. 4 shows a block diagram of an overview of graphene ink with 2-5% w/v solids content of one embodiment. FIG. 4 shows a graphene ink with 2-5% w/v solids content 400. Wherein w/v stands for weight by volume (w/v) and solids content comprises the van der Waal material, such as graphene and the polymer binder. The graphene ink includes a predetermined quantity of hydrophobic fluid 410. Another component is a polymer binder dispersed in hydrophobic fluid at a w/v concentration of 2-5% 420. Oxidized graphene dispersed in hydrophobic fluid at a w/v concentration of 2-5% 430. Other components are included to combine ink additives at a w/v concentration of <5% or <10% or <20% or <30% or up to 98% with oxidized graphene dispersion 440. It shall be understood that the ink additives can also be combined with the polymer binder dispersion or be combined with the graphene ink with 2-5% w/v solids content. In another embodiment, the mixed chemicals include combining polymer dispersion and graphene dispersion at a ratio of at least one of 3:1, 3:2, and 1:1 450. One exemplary embodiment, includes a drying step whereby the graphene dispersion and polymer binder dispersion are combined, the ink is dried under a vacuum to evaporate a portion or all of one or more hydrophobic fluids used in the dispersions. The specified drying temperature can range between 100 C and 300 C. A graphene ink with 2-5% w/v solids content 460 meets predetermined characteristics, including density 470, surface tension 472, viscosity 474, particle size 476, and zeta potential 478 of one embodiment.

The exemplary ink formula disperses a van der Waal material, including oxidized graphene, in a hydrophobic fluid at a w/v concentration between 2% and 5% and combines the said graphene dispersion with a polymer binder dispersed in a hydrophobic fluid at a w/v concentration between 2% and 5%. The two dispersions, the graphene dispersion and the polymer binder dispersion, are combined at a ratio of at least one of 3:1, 3:2, and 1:1.

In another exemplary embodiment, the graphene dispersion is combined with additives, including dispersing agents or wetting agents and other additives at a w/v concentration of less than or equal to 5%. It should be appreciated that some exemplary embodiments may not require additives. In other exemplary embodiments, the additives can be combined with the ink after combining the two dispersions, graphene dispersion and polymer binder dispersion at a ratio of at least one of 4:1, 4:2 and 1:1. Exemplary ink additives include dispersing agent, rheology modifiers, and surfactants, plasticizers, cross-linking agents, conductivity improvers, antifoaming agents, UV stabilizers, flame retardants and adhesion promoters. In other exemplary embodiments, all the ink ingredients, such as oxidized graphene, hydrophobic fluids, polymer binders, and additives, are combined in a single step, mixed using at least one of various mixing techniques such as sonication, shear-mixing, planetary mixing, or hand mixing.

Exemplary hydrophobic fluids include alcohols, terpenes, nonpolar solvents, organic solvents, hydrocarbons, silicone oils, fluorinated fluids, chlorinated fluids, surfactants, and blends of various hydrophobic fluids. Hydrophobic fluids are used to disperse oxidized graphene and polymer binders at a concentration between 2% and 5%, between 5% and 10%, between 10% and 20% and even up to 98%. It shall be understood that the ink additives can be combined with the polymer binder dispersion or combined with the graphene ink with 2-5% w/v solids content.

Exemplary polymer binders include any polymer binders, including cellulose derivatives, polyvinylpyrrolidone (PVP), polyvinyl alcohol (PVA), polyethylene glycol (PEG), polyurethane (PU), polystyrene (PS), acrylic polymers, epoxy resins, and polyimides. The polymer binder dispersion is combined with the oxidized graphene dispersion at a ratio of at least one of 4:1, 4:2, and 1:1. One exemplary embodiment includes a drying step whereby the after the graphene dispersion and polymer binder dispersion are combined, the ink is dried under a vacuum to evaporate a portion or all of one or more hydrophobic fluids used in the dispersions. The specified drying temperature can range between 100° C. and 300° C. The combined mixture results in a graphene ink with a 2-5% w/v solids content. The resulting density and surface tension of the ink is representative of the ink's viscosity, particle size, and zeta potential.

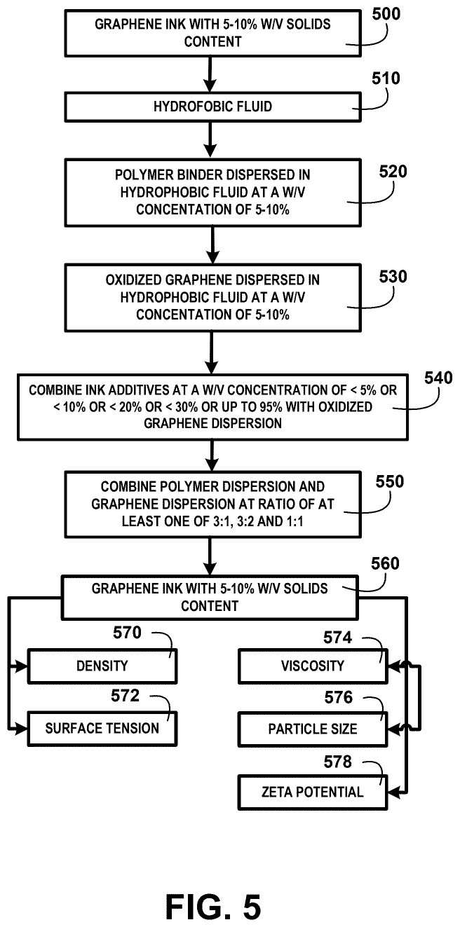

FIG. 5 shows a block diagram of an overview of graphene ink with 5-10% w/v solids content of one embodiment. FIG. 5 shows a graphene ink with 5-10% w/v solids content 500. The graphene ink includes a predetermined quantity of hydrophobic fluid 510. Polymer binder dispersed in hydrophobic fluid at a w/v concentration of 5-10% 520. Oxidized graphene dispersed in hydrophobic fluid at a w/v concentration of 5-10% 530. Other components are included to combine ink additives at a w/v concentration of <5% or <10% or <20% or <30% or up to 95% with oxidized graphene dispersion 540. It shall be understood that the ink additives can also be combined with the polymer binder dispersion or combined with the graphene ink with 5-10% w/v solids content. In another embodiment, the mixed chemicals include a combination of polymer dispersion and graphene dispersion at a ratio of at least one of 3:1, 3:2, and 1:1 550. One exemplary embodiment includes a drying step whereby the graphene dispersion and polymer binder dispersion are combined, and the ink is dried under a vacuum to evaporate a portion or all of one or more hydrophobic fluids used in the dispersions. The specified drying temperature can range between 100° C. and 300° C.

A graphene ink with 5-10% w/v solids content 560, meet predetermined characteristics, including density 570, surface tension 572, viscosity 574, particle size 576, and zeta potential 578 of one embodiment.

The exemplary ink formula disperses a van der Waal material that includes oxidized graphene in a hydrophobic fluid at a w/v concentration between 5% and 10% and combines the said graphene dispersion with a polymer binder dispersion consisting of a hydrophobic fluid at a w/v concentration between 5% and 10%. The two dispersions, the graphene dispersion and the polymer binder dispersion, are combined at a ratio of at least one of 3:1, 3:2, and 1:1.

In another exemplary embodiment, the graphene dispersion is combined with additives, including dispersing agents or wetting agents and other additives at a w/v concentration of less than or equal to 95%. It should be appreciated that some exemplary embodiments may not require additives. In other exemplary embodiments, the additives can be combined with the ink after combining the two dispersions, graphene dispersion and polymer binder dispersion at a ratio of at least one of 3:1, 3:2 and 1:1. In other embodiments, the additives can be combined with the polymer binder dispersion at various concentrations so that the total concentration of the graphene ink is equal to 100%. Exemplary ink additives include dispersing agents, rheology modifiers, surfactants, plasticizers, cross-linking agents, conductivity improvers, antifoaming agents, UV stabilizers, flame retardants, and adhesion promoters. In other exemplary embodiments, all the ink ingredients, such as oxidized graphene, hydrophobic fluids, polymer binders, and additives, are combined in a single step, mixed using at least one of various mixing techniques such as sonication, shear-mixing, planetary mixing, or hand mixing.

Exemplary hydrophobic fluids include alcohols, terpenes, nonpolar solvents, organic solvents, hydrocarbons, silicone oils, fluorinated fluids, chlorinated fluids, surfactants, and blends of various hydrophobic fluids. Hydrophobic fluids are used to disperse oxidized graphene and polymer binders at a concentration between 5% and 10%.

Exemplary polymer binders include any polymer binder including cellulose derivatives, polyvinylpyrrolidone (PVP), polyvinyl alcohol (PVA), polyethylene glycol (PEG), polyurethane (PU), polystyrene (PS), acrylic polymers, epoxy resins, and polyimides. The polymer binder dispersion is combined with the oxidized graphene dispersion at a ratio of at least one of 4:1, 4:2, and 1:1. The combined mixture results in a graphene ink with a 5-10% w/v solids content. The resulting density and surface tension of the ink is representative of the ink's viscosity, particle size, and zeta potential.

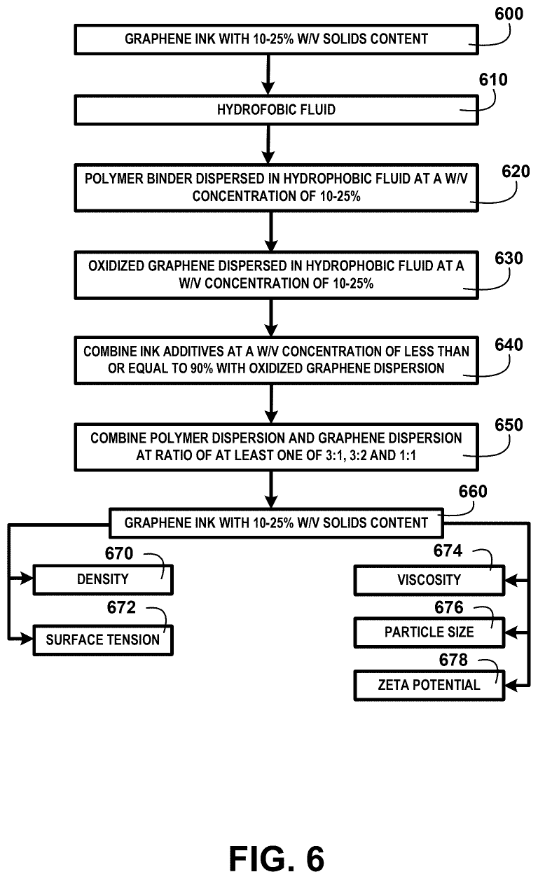

FIG. 6 shows a block diagram of an overview of graphene ink with 10-25% w/v solids content of one embodiment. FIG. 6 shows a graphene ink with 10-25% w/v solids content 600. The graphene ink includes a predetermined quantity of hydrophobic fluid 610. Polymer binder dispersed in hydrophobic fluid at a w/v concentration of 10-25% 620. Oxidized graphene dispersed in hydrophobic fluid at a w/v concentration of 10-25% 630. Other components are included to combine ink additives at a w/v concentration of less than or equal to 90% with oxidized graphene dispersion 640. It shall be understood that the ink additives can also be combined with the polymer binder dispersion or combined with the graphene ink with 10-25% w/v solids content. In another embodiment, the mixed chemicals include a combined polymer dispersion and graphene dispersion at a ratio of at least one of 3:1, 3:2, and 1:1 650. One exemplary embodiment, includes a drying step whereby the graphene dispersion and polymer binder dispersion are combined, and the ink is dried under a vacuum to evaporate a portion or all of one or more hydrophobic fluids used in the dispersions. The specified drying temperature can range between 100° C. and 300° C. The graphene ink with 10-25% w/v solids content 660 meet predetermined characteristics, including density 670, surface tension 672, viscosity 674, particle size 676, and zeta potential 678 of one embodiment.

The exemplary ink formula disperses a van der Waal material, including oxidized graphene, in a hydrophobic fluid at a w/v concentration between 10% and 25% and combines the said graphene dispersion with a polymer binder dispersed in a hydrophobic fluid at a w/v concentration between 10% and 25%. The two dispersions, the graphene dispersion and the polymer binder dispersion, are combined at a ratio of at least one of 4:1, 4:2, and 1:1.

In another exemplary embodiment, the graphene dispersion is combined with additives, including dispersing agents or wetting agents, and others at a w/v concentration of less than or equal to 90%. It should be appreciated that some exemplary embodiments may not require additives. In other exemplary embodiments, the additives can be combined with the polymer binder dispersion or the ink after combining the two dispersions, graphene dispersion and polymer binder dispersion at a ratio of at least one of 4:1, 4:2 and 1:1. Exemplary ink additives include dispersing agent, rheology modifiers, and surfactants, plasticizers, cross-linking agents, conductivity improvers, antifoaming agents, UV stabilizers, flame retardants and adhesion promoting other exemplary embodiments, all the ink ingredients, such as oxidized graphene, hydrophobic fluids, polymer binders, and additives, are combined in a single step, mixed using at least one of various mixing techniques such as sonication, shear-mixing, planetary mixing, or hand mixing.

Exemplary hydrophobic fluids include hydrophobic fluids include alcohols, terpenes, nonpolar solvents, organic solvents, hydrocarbons, silicone oils, fluorinated fluids, chlorinated fluids, surfactants, and blends of various hydrophobic fluids. Hydrophobic fluids are used to disperse oxidized graphene and polymer binders at a concentration between 10% and 25%.

Exemplary polymer binders include any polymer binder including cellulose derivatives, polyvinylpyrrolidone (PVP), polyvinyl alcohol (PVA), polyethylene glycol (PEG), polyurethane (PU), polystyrene (PS), acrylic polymers, epoxy resins, and polyimides. The polymer binder dispersion is combined with the oxidized graphene dispersion at a ratio of at least one of 3:1, 3:2, and 1:1. The combined mixture results in a graphene ink with a 10-25% w/v solids content. The resulting density and surface tension of the ink is representative of the ink's viscosity, particle size, and zeta potential.

Creating a graphene ink that has desirable characteristics, including stability, conductivity, and printability, requires a careful balance of oxidized graphene, hydrophobic fluid, polymer binder, and additives.

The oxidized graphene component of the ink provides conductive and mechanical properties of the ink. Oxidized graphene introduces functional groups (e.g., hydroxyl, carboxyl, epoxy groups, etc.). The functional groups improve dispersibility in various solvents. In one embodiment, plasma-produced graphene is used. Plasma-produced graphene involves using a high-energy state of matter (plasma) to facilitate the synthesis and exfoliation of graphene from graphite or other carbon sources. The method has several advantages, including high purity, fewer defects, uniform thin layers, and overall control over the properties of the graphene produced. Plasma methods are also highly reproducible and scalable. In another embodiment, reduced graphene oxide (rGO) is used. rGO is produced by removing oxygen-containing functional groups, restoring the graphene structure, and enhancing its electrical and mechanical properties. In another embodiment, graphene oxide (GO) is used. GO is produced through various methods involving the oxidation of graphite. Such methods are shown in FIG. 2.

In another embodiment, holey graphene is used. Holey graphene refers to graphene sheets that have been intentionally perforated with a network of nanometer-sized holes. These holes can be introduced through various chemical, physical, or lithographic processes, resulting in a structure that retains the mechanical strength and electrical conductivity of graphene while also gaining new properties due to the presence of the holes.

In another embodiment, single-layer graphene is used. Single-layer graphene has sp2 hybridized carbon atoms that form a strong covalent bond with three neighboring carbon atoms. This arrangement creates a hexagonal pattern that extends in a two-dimensional plane. The fourth electron of each carbon atom is free to move within the plane, contributing to the material's excellent electrical conductivity. In yet another embodiment, multi-layer graphene is used. Multi-layer graphene refers to a material composed of more than one, but typically fewer than ten, layers of graphene stacked on top of each other. Each layer in multi-layer graphene is a single sheet of carbon atoms arranged in a hexagonal lattice, like single-layer graphene, but the presence of multiple layers can significantly alter its properties. In still another embodiment, functionalized graphene is used. Functionalized graphene is chemically modified to introduce various functional groups or molecules onto its surface or edges. This process of functionalization alters the chemical, physical, and electronic properties of graphene, making it more versatile for a wide range of applications, including sensors and energy storage, among other applications.

Hydrophobic fluid is used to improve dispersion stability and tailor the ink's rheological properties. Van der Waal materials, including graphene, are inherently hydrophobic due to their nonpolar nature. Using hydrophobic solvents can improve the compatibility between the solvent and graphene flakes, leading to better dispersion. This reduces the tendency of graphene to agglomerate or restack, ensuring a stable, uniform ink. Hydrophobic fluids can include organic solvents, nonpolar solvents, surfactants, or a blend of all three. Hydrophobic fluids also include alcohols, terpenes, hydrocarbons, silicone oils, fluorinated fluids, chlorinated fluids, and blends of two or more hydrophobic fluids.

Polymer binders provide dispersion stabilization, film formation, control of ink properties, and offer additional functional properties. The binders also help prevent graphene from aggregating due to van der Waals forces.

Polymer binders adsorb onto the graphene surface, providing steric hindrance or electrostatic repulsion that prevents the flakes from aggregating. This maintains a stable, uniform dispersion in the ink. Polymer binders improve the adhesion of graphene flakes to substrates, which is essential for forming durable coatings or films. This is particularly important in applications including printed electronics, where good adhesion ensures the longevity and reliability of the printed patterns. The addition of polymer binders affects the viscosity and rheological properties of the ink, which are tailored for various printing techniques (e.g., inkjet, screen, pad, or flexographic printing, spin coating, spray coating, etc.). The right viscosity ensures smooth ink flow, proper deposition, and consistent pattern formation. In one embodiment, polymer binders include: cellulose derivatives, polyvinylpyrrolidone (PVP), polyvinyl alcohol (PVA), polyethylene glycol (PEG), polyurethane (PU), polystyrene (PS), acrylic polymers, epoxy resins, and polyimides.

Ink additives are substances added to inks to modify or improve specific properties, including stability, printability, and functionality. Dispersing agents assist in uniformly distributing graphene flakes throughout the solvent, preventing settling and aggregation. Rheological modifiers adjust the viscosity of the ink to suit specific printing techniques, including inkjet, screen, pad, spray/spin coating, or flexographic printing, etc. Surfactants reduce surface tension and help stabilize the dispersion of graphene flakes in the solvent. Plasticizers increase the flexibility and durability of printed films. Cross-linking agents are chemicals that facilitate the formation of covalent bonds between polymer chains or between polymer chains and other molecules. This process, known as cross-linking, results in a network structure that can significantly alter the physical, mechanical, and chemical properties of the material. Conductivity enhancers improve the electrical conductivity of the printed graphene film. Antifoaming agents prevent or reduce foam formation during ink preparation and application. UV stabilizers or other types of curing agents facilitate the curing process of the ink, especially in formulations requiring UV or thermal curing. Flame retardants are chemicals added to materials to inhibit or slow down the spread of fire. They work by interfering with the combustion process at various stages: ignition, flame spread, and smoke production. Adhesion promoters are chemicals or materials added to a formulation to improve the adhesion between different layers or materials. They work by creating a strong bond at the interface, improving the overall durability and performance of coatings, adhesives, composites, and inks.

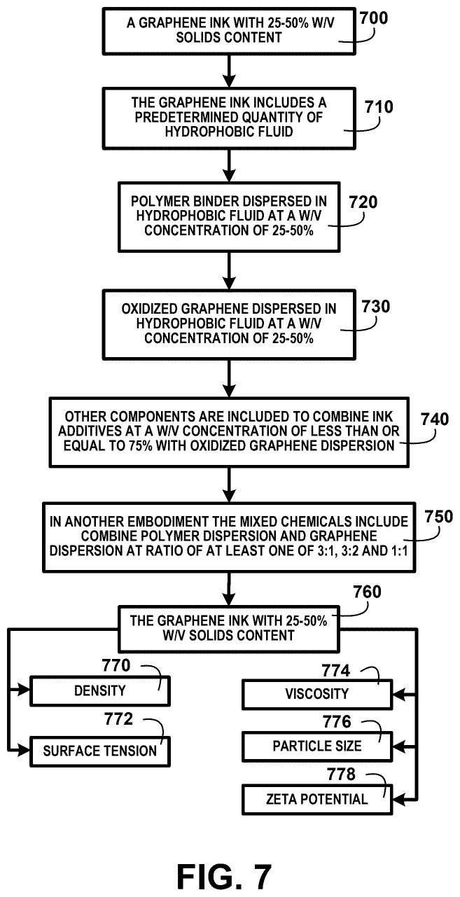

FIG. 7 shows a block diagram of an overview of graphene ink with 25-50% w/v solids content of one embodiment. FIG. 7 shows a graphene ink with 25-50% w/v solids content 700. The graphene ink includes a predetermined quantity of hydrophobic fluid 710. Polymer binder dispersed in hydrophobic fluid at a w/v concentration of 25-50% 720. Oxidized graphene dispersed in hydrophobic fluid at a w/v concentration of 25-50% 730. Other components are included to combine ink additives at a w/v concentration of less than or equal to 75% with oxidized graphene dispersion 740. It shall be understood that the ink additives can also be combined with the polymer binder dispersion or combined with the graphene ink with 25-50% w/v solids content. In another embodiment, the mixed chemicals include a combination of polymer dispersion and graphene dispersion at a ratio of at least one of 3:1, 3:2, and 1:1 750. One exemplary embodiment, includes a drying step whereby the graphene dispersion and polymer binder dispersion are combined, the ink is dried under a vacuum to evaporate a portion or all of one or more hydrophobic fluids used in the dispersions. The specified drying temperature can range between 100° C. and 300° C. The graphene ink with 25-50% w/v solids content 760 meet predetermined characteristics, including density 770, surface tension 772, viscosity 774, particle size 776, and zeta potential 778 of one embodiment.

The exemplary ink formula disperses a van der Waal material, including oxidized graphene, in a hydrophobic fluid at a w/v concentration between 25% and 50% and combines the said graphene dispersion with a polymer binder dispersed in a hydrophobic fluid at a w/v concentration between 25% and 50%. The two dispersions, the graphene dispersion and the polymer binder dispersion, are combined at a ratio of at least one of 4:1, 4:2, and 1:1.

In another exemplary embodiment, the graphene dispersion is combined with additives, including dispersing agents or wetting agents, and others at a w/v concentration of less than or equal to 75%. It should be appreciated that some exemplary embodiments may not require additives. In other exemplary embodiments, the additives can be combined with the ink after combining the two dispersions, graphene dispersion and polymer binder dispersion at a ratio of at least one of 4:1, 4:2 and 1:1. Exemplary ink additives include dispersing agent, rheology modifiers, surfactants, plasticizers, cross-linking agents, conductivity improvers, antifoaming agents, UV stabilizers, flame retardants and adhesion promoters. In other exemplary embodiments, all the ink ingredients, such as oxidized graphene, hydrophobic fluids, polymer binders, and additives, are combined in a single step, mixed using at least one of various mixing techniques such as sonication, shear-mixing, planetary mixing, or hand mixing.

Exemplary hydrophobic fluids include alcohols, terpenes, nonpolar solvents, organic solvents, hydrocarbons, silicone oils, fluorinated fluids, chlorinated fluids, surfactants, and blends of various hydrophobic fluids. Hydrophobic fluids are used to disperse oxidized graphene and polymer binders at a concentration between 25-50% w/v.

Exemplary polymer binders include any polymer binder, including cellulose derivatives, polyvinylpyrrolidone (PVP), polyvinyl alcohol (PVA), polyethylene glycol (PEG), polyurethane (PU), polystyrene (PS), acrylic polymers, epoxy resins, and polyimides. The polymer binder dispersion is combined with the oxidized graphene dispersion at a ratio of at least one of 3:1, 3:2, and 1:1. The combined mixture results in a graphene ink with a 10-25% w/v solids content. The resulting density and surface tension of the ink is representative of the ink's viscosity, particle size, and zeta potential.

In one embodiment, plasma-produced graphene is used. Methods of using plasma to produce graphene include thermal plasma, cold plasma, and laser-induced plasma. Thermal plasma uses high temperatures to generate plasma, which exfoliates graphite into graphene. Common methods of thermal plasma include arc discharge, plasma jet, induction thermal plasma, and plasma torch. The arc discharge method involves generating a plasma arc between two graphite electrodes submerged in an inert gas atmosphere. The high temperature of the arc (˜4000° C.) vaporizes the graphite, and the subsequent cooling leads to the formation of graphene. The plasma jet method uses a plasma torch to generate a high-temperature jet of plasma that can exfoliate graphite into graphene sheets. Induction thermal plasma uses radio frequency (RF) or microwave energy to generate a plasma field. The high-energy plasma is used to exfoliate graphite into graphene. The plasma torch method uses a direct current (DC) to create a high-temperature plasma arc that can exfoliate and reduce graphite to graphene. These methods for producing oxidized graphene using thermal plasma methods are efficient, scalable, and consistently produce few-layer or single-layer graphene. Cold plasma generates plasma at lower temperatures, often using ionized gas containing a significant number of reactive species (electrons, ions, radicals). These reactive species interact with graphite or other carbon sources to facilitate the exfoliation and reduction processes necessary for producing graphene. Methods of cold plasma production include Plasma-enhanced Chemical Vapor Deposition (PECVD), Plasma-enhanced Exfoliation, and Plasma Arc Discharge in Solution. PECVD is suitable for temperature-sensitive substrates and produces high-quality, uniform graphene films. PECVD uses a cold plasma generated from a gas mixture that contains a carbon source (e.g., methane, etc.) and a carrier gas (e.g., hydrogen, argon, etc.). The carbon-containing gas is dissociated in the plasma, and carbon atoms are deposited onto the substrate, forming graphene layers. Plasma-induced exfoliation not only exfoliates the graphite but also helps reduce any oxygen-containing functional groups. Graphite powder is introduced to a cold plasma reactor and exposed to a cold plasma generated by gases like argon, hydrogen, or a mixture of the two. The reactive species in the plasma induce exfoliation of the graphite layer, producing graphene. Plasma Arc Discharge in Solution is suitable for large-scale production and is performed under ambient conditions without high temperatures. Graphite is dispersed in a suitable solvent, and a plasma arc is generated within the solution using electrodes. The cold plasma interacts with the graphite particles, causing exfoliation. Laser-induced plasma (LIP) is generated by the interaction of a high-intensity laser pulse with a material. This interaction causes the material to ionize and form a plasma. Methods of laser-induced plasma production include Laser-Induced Breakdown Spectroscopy (LIBS), Laser-Induced Fluorescence (LIF), Laser-Induced Plasma-Assisted Ablation (LIPAA), and Laser-Induced Plasma for Nanoparticle Synthesis. LIBS is a type of laser-induced plasma spectroscopy where a high-intensity laser pulse is focused on a sample to generate a plasma. LIF is a technique where a laser is used to excite atoms or molecules in the plasma, causing them to emit light (fluorescence). LIPAA is a process where a laser-induced plasma is used to assist in the ablation of materials. Laser-induced plasma produces nanoparticles when the high energy of the laser pulse vaporizes the material, and the plasma aids in the formation of nanoparticles as the vapor cools.

In another embodiment, reduced graphene oxide (rGO) is used. Methods of reducing oxygen-containing functional groups and enhancing conductivity and mechanical properties include chemical reduction, thermal reduction, hydrothermal and solvothermal reduction, and electronic reduction. Chemical reduction uses reducing agents to remove oxygen-containing functional groups. Common reducing agents include hydrazine hydrate (N2H4), sodium borohydride (NaBH4), ascorbic acid (vitamin C), hydrochloric acid (HI), zinc powder (Zn), and hydroxylamine (NH2OH). Hydrazine hydrate reduces GO by donating electrons, which convert the oxygen functional groups back into carbon or hydrogen. GO is dispersed in water, and hydrazine hydrate is added. The mixture is heated to 80-100° C. for several hours. Sodium borohydride (NaBH4) acts as a reducing agent by transferring hydride ions to the oxygen groups on GO. GO is dispersed in an aqueous or alcoholic solution, and NaBH₄ is added at room temperature or with mild heating. Ascorbic acid reduces GO by donating electrons and converting oxygen functional groups to water, resulting in rGO. GO is dispersed in water, and ascorbic acid is added. The mixture is stirred at room temperature or slightly elevated temperatures. Hydroiodic acid (HI) reduces GO by donating protons and electrons, effectively removing oxygen groups. GO is treated with HI at elevated temperatures (e.g., 40-100° C.) for a few hours. Zinc (Zn) powder reduces GO by acting as an electron donor in an acidic medium. GO is dispersed in water with zinc powder and an acid (e.g., hydrochloric acid). The mixture is stirred and heated. Hydroxylamine (NH2OH) reduces GO by donating electrons and converting oxygen groups into hydroxyl and amino groups. GO is dispersed in water, and hydroxylamine is added. The mixture is heated to around 90° C. Thermal reduction involves heating GO to a high temperature to remove oxygen-containing groups. This process restores the graphene structure and improves its electrical conductivity and mechanical properties. Thermal annealing is done in a furnace at temperatures ranging from 200° C. to 1100° C. The annealing process can be carried in an inert atmosphere (e.g., argon, nitrogen) or a reducing atmosphere (e.g., hydrogen). The duration of the heating can vary from a few minutes to several hours, depending on the desired degree of reduction. Another method, rapid thermal annealing (RTA), involves rapidly heating GO to temperatures ranging from 700° C. to 1000° C. The process lasts from a few seconds to a few minutes, allowing for quick reduction without significant structural damage. RTA can be carried out in an inert or reducing atmosphere. Another method, flash reduction, uses a sudden burst of high-temperature heat, including Joule heating or laser irradiation, to reduce GO. Another method, microwave-assisted thermal reduction, uses microwave radiation to rapidly heat in the range of 200° C. to 400° C. and reduce GO. Reduction occurs within a few minutes. Another method, thermal shock reduction, involves subjecting GO to sudden and extreme temperatures for a short time. GO is preheated to a moderate temperature and then exposed to a very high temperature (e.g., 1000° C.) for a very short time (seconds). It should be appreciated that thermal reduction methods shall not be limited to those specified. All thermal reduction methods are effective at converting GO to rGO. The choice of method depends on the desired reduction level, processing time, and equipment availability.

In another embodiment, graphene oxide is used. The most common method, Hummers' Method, uses strong oxidizing agents. GO powder is mixed with sodium nitrate with concentrated sulfuric acid (H₂SO₄) in an ice bath. Then, potassium permanganate (KMnO₄) is slowly added while maintaining the temperature below 20° C. The temperature is increased to 45-40° C. and stirred for several hours. The solution is diluted with water, and hydrogen peroxide (H₂O₂) is added. Lastly, the solution is filtered, washed, and dried. Modified Hummers' Method is another approach that improves safety, yield, and efficiency. The Modified Hummers' Method optimizes reaction times and temperatures to improve oxidation efficiency by eliminating sodium nitrate and reducing toxic gas formation. Brodie's Method is another approach that involves the use of fuming nitric acid and potassium chlorate to oxidize graphite. First, graphite is mixed with fuming nitric acid, and potassium chlorate is added slowly to the mixture. Next, the reaction mixture is diluted with water, and the product is filtered and washed with water and hydrochloric acid to remove residual acids and chlorate. The Staudenmaier Method, is another method used to improve reaction control and safety. First, graphite is added to a mixture of sulfuric acid and nitric acid. Next, potassium chlorate is added slowly while stirring. A purification step follows. The reaction mixture is diluted and filtered. The product is washed to remove acids and chlorate. Another approach to producing GO is the Tour's Method. The Tour's Method is a refinement of the Hummers' Method designed to increase the yield and efficiency of the graphene oxide production process. First, graphite is mixed with sulfuric acid. Then, potassium permanganate is added gradually while keeping the mixture cold. The mixture is heated to 50° C. and stirred. Hydrogen peroxide is added to stop the reaction. Next, the mixture is washed and filtered to remove residual chemicals and obtain graphene oxide. Electronic Exfoliation is another method of producing GO that involves applying an electric potential to graphite electrodes submerged in an electrolyte solution, which induces the intercalation of ions into the graphite layers, leading to exfoliation. The process is relatively straightforward, scalable, and environmentally friendly compared to chemical methods. First, electronic cell electrodes are prepared. The graphite electrode acts as an anode, and a counter electrode serves as the cathode. Both electrodes are immersed in an electrolyte solution. A constant voltage is applied (typically 5-10 V) between the anode and cathode. During the process, ions from the electrolyte intercalate into the graphite layers at the anode, causing the layers to separate and exfoliate into graphene oxide sheets. Gas evolution (e.g., oxygen bubbles) at the anode surface can assist in the exfoliation process by generating mechanical forces that further separate the layers. The exfoliated graphene oxide sheets will disperse in the electrolyte solution, forming a colloidal suspension. The suspension is filtered and washed with deionized water to remove impurities. Lastly, the GO is dried at a suitable temperature (˜60° C.).

In another embodiment, holey graphene is used. Holey graphene is a nonporous graphene that has intentionally created holes or pores. These perforations can vary in size, shape, and distribution as the perforations are produced using different methods. One method of the exemplary embodiment is chemically etched holey graphene, which creates holes by selectively removing carbon atoms using chemical reagents. The hole size produced typically ranges from a few nanometers to tens of nanometers and can be controlled by varying the etching time and concentration of the reagents. The exemplary method also includes chemical oxidation, using strong oxidizing agents like potassium permanganate (KMnO₄) in an acidic environment to etch holes into graphene oxide. Another exemplary method is plasma treatment, which exposes graphene to oxygen plasma to etch the nanoscale holes. Another method of the exemplary embodiment is laser-induced ablation of holey graphene. This method creates holes in the graphene by locally heating and ablating the material. The method is finely controlled by adjusting laser power, pulse duration, and scanning speed, resulting in high precision in hole placement and size. Exemplary methods of laser-induced holey graphene include, but are not limited to, laser ablation and photothermal etching. Laser ablation uses focused laser beams to ablate the graphene sheets and create holes. Photothermal etching uses laser pulses to locally increase the temperature and selectivity to remove carbon atoms. Another method of the exemplary embodiment is electronic etching, which involves applying an electric potential to graphene in an electrolyte solution to create uniform holes. Exemplary methods of electronic etching include, but are not limited to, electronic oxidation and electronic bubble formation. Electronic oxidation applies a voltage in an electrolyte solution to oxidize and remove carbon atoms from the graphene sheet. Electronic bubble formation uses gas bubbles generated at the electrode surface to physically disrupt and create holes in the graphene. In both methods, hole size and uniformity can be controlled by adjusting voltage, electrolyte concentration, and etching time. Another method of the exemplary embodiment is template-assisted methods. This method uses templates or masks to protect certain areas of the graphene, while the exposed areas are etched to create holes. This method allows for precise control over the pattern and arrangement of holes. Exemplary masking types of template-assisted methods include, but are not limited to, block copolymer templates and metal nanoparticle templates. Another exemplary method is self-assembly techniques that utilize the natural tendency of certain molecules or particles to former ordered structures used to create holey graphene. Block copolymers are self-assembled into well-defined nanostructures that serve as masks during the etching process. Colloidal particles are used to form a mask on the graphene surface. The spaces between the particles are etched to create holes, and then the particles are removed. Another method of the exemplary embodiment is ion beam milling. This method uses focused ion beams to sputter away carbon atoms from graphene to create holes. This method can be controlled with high precision, ranging from a few nanometers to micrometers, and creates complex and highly precise patterns of holes. Gallium ions are used for their high-precision milling and are suitable for intricate patterns and small-size features. Electron Beam Lithography (EBL) is another exemplary method, which uses a focused beam of electrons to define patterns on a resist-coated graphene surface. The exposed areas are etched to create holes. EBL provides high resolution, making it ideal for creating very small and precise holes. Mechanical Methods is another exemplary embodiment that involves physically removing material to create holes in the graphene structure. Nanoimprint lithography uses a mold with a nanopattern that is pressed into a resist-coated graphene sheet. The pattern is transferred to the resist and then etched to create holes.

The types of hydrophobic fluid for graphene ink dispersions depend on the desired stability, drying time, uniform film formation, enhancing electrical properties, chemical compatibility, and substrate compatibility. Hydrophobic fluid interacts favorably with the hydrophobic surface of graphene flakes, reducing the tendency for graphene flakes to aggregate. This interaction helps maintain a stable, uniform dispersion of graphene in the solvent. Hydrophobic fluid also improves wetting and adhesion of graphene dispersions on hydrophobic substrates (e.g., ceramics, plastics, polymers, paper, glass, and non-polar surfaces). This compatibility ensures uniform coating and strong adherence to the graphene layer. Hydrophobic fluids have specific evaporation rates that can be tailored to achieve uniform coating and film formation. Hydrophobic fluids also influence the viscosity of the ink dispersion, enabling its use for deposition methods like inkjet printing, aerosol printing, spray/spin coating, dip-coating, pad printing, and screen printing, among other methods of deposition. Hydrophobic fluids also tend to leave fewer polar residues upon evaporation compared to hydrophilic solvents. This results in cleaner graphene films with better electrical conductivity and fewer defects. Hydrophobic fluid also reduces the risk of graphene oxidation during processing and preserving its electrical properties. Hydrophobic fluids also dissolve other hydrophobic additives, including surfactants, functionalizing agents, polymers, and other materials used to stabilize the graphene ink dispersion. Alcohols, glycols, terpenes, terpene alcohols, non-polar solvents, organic solvents, hydrocarbons, silicone oils, fluorinated solvents, chlorinated solvents, surfactants, and blends of these are among the types of hydrophobic fluids to be used in the exemplary graphene ink.

An exemplary fluid with hydrophobic characteristics used in a graphene ink dispersion is alcohol. While alcohols are not typically considered hydrophobic fluids because they have polar hydroxyl groups that make them hydrophilic. However, some higher molecular weight alcohols or branched alcohols exhibit more hydrophobic characteristics due to their larger non-polar alkyl groups. These are used in exemplary formulations of graphene ink dispersions to achieve a balance between hydrophobic and hydrophilic properties. In one exemplary embodiment, butanol (n-butyl Alcohol), a moderately hydrophobic fluid due to its longer alkyl chain, is used. In another exemplary embodiment, Hexanol (n-Hexyl Alcohol), a more hydrophobic substance than butanol, is used to provide better solubility for non-polar substances. Octanol (n-Octyl Alcohol), a higher hydrophobicity fluid owning to its even longer alkyl chain, making it less polar and more compatible with non-polar materials, is used in another exemplary embodiment. Isobutanol (Isobutyl Alcohol), a moderately hydrophobic fluid that has reduced polarity, is used in an exemplary embodiment of graphene in. Other alcohols that are used in exemplary embodiments include ethyl alcohol, isopropyl alcohol, and methanol. Alcohol can also be blended with other alcohols and other solvents, including glycols. Exemplary alcohol blends combine different types of alcohol embodiments including ethanol and isopropanol blend, isopropanol blend, ethanol and butanol blend, isopropanol and propylene glycol blend, propylene glycol blend, and polyethylene glycol (PEG) blend. Alcohols to blend with PEG are ethanol, isopropanol, methanol, and butanol. While most alcohols are hydrophilic, certain higher molecular weight alcohols are used to balance stability, enhancing wetting on hydrophobic substrates and tailoring the ink viscosity to specific material deposition methods.

Terpenes are another class of hydrophobic fluid used in the exemplary graphene ink. Terpenes are a diverse class of organic compounds built from isoprene units (C5H8). There are six general types of terpenes: Monoterpenes (C10H16), Sesquiterpenes (C15H24), Diterpenes (C20H42), Triterpenes (C40H48), Tetraterpenes (C40H64), and Polyterpenes (C5H8) n, with Monoterpenes (C10H16) used in the exemplary embodiments. Exemplary Monoterpenes are Limonene, Pinene, Myrcene, Terpinene, Terpineol, Caryophyllene, and Linalool. Limonene is a non-polar, low-boiling-point (176° C.) terpene that is used to disperse organic compounds. Limonene's benefits include low toxicity and good dispersibility. Pinene, a bicyclic monoterpene, which exists in two structural isomers α-pinene and β-pinene is used for its non-polar characteristics and low boiling point (155-156° C.). Another terpene, Myrcene is an acyclic monoterpene that also has a low boiling point (167° C.) is used. In another exemplary embodiment, Terpinene is used in several isomeric forms including α-terpinene, γ-terpinene, and δ-terpinene, and has boiling points ranging from 170-190° C. Caryophyllene, a bicyclic sesquiterpene, that has a higher boiling point of 266° C. is used for slower evaporation and improved film formation. Linalool, another terpene that has a boiling point of around 198-199° C. is used in another exemplary embodiment. In other exemplary embodiments, terpenes are blended to make terpene blends that are used as the hydrophobic fluid in the graphene ink. Exemplary embodiments of terpene alcohol blends are Limonene and Ethanol Blend, Pinene and Isopropanol Blend, Terpineol and Propylene Glycol Blend, Myrcene and Butanol Blend. These blends combine the best properties of both terpenes and alcohols, creating a graphene ink formula with optimal solubility, stability, and viscosity.

Non-polar fluid is another class of hydrophobic fluid used in the exemplary graphene ink. These fluids have unique properties including high solvating power, making them suitable for dispersing graphene inks. Chloroform (Trichloromethane) is one exemplary non-polar fluid used for its excellent solvating power and low boiling point of 61° C. Other non-polar fluids used in exemplary embodiments are Tetrachloroethylene (Perchloroethylene) with a boiling point of 121° C.; Dichloromethane (Methylene Chloride) with a boiling point of 40° C.; Carbon Tetrachloride with a boiling point of 76° C.; Diethyl Ether (Ethyl Ether) with a boiling point of 44° C.; Tetrahydrofuran (THF) with a boiling point of 66° C. and Acetone with a boiling point of 56° C.

The organic solvent is another class of hydrophobic fluid used in exemplary graphene ink to stabilize and disperse graphene flakes effectively. Organic solvents with high solvating power and included in exemplary embodiments are N-Methyl-2-pyrrolidone (NMP), Dimethylformamide (DMF), Dimethyl Sulfoxide (DMSO), Ethylene Glycol and Propylene Glycol. In one exemplary embodiment, NMP is used for its excellent solvating power and film formation at high curing temperatures (202° C.). The high curing temperature allows for controlled evaporation, ensuring uniform film formation. In another exemplary embodiment, DMF is used to create stable, high-concentration graphene ink dispersions. With a lower boiling point (154° C.), it is used with substrates that have lower temperature tolerances including PET and other polymer substrates. In another exemplary embodiment, DMSO is used to effectively dissolve and disperse graphene and is non-toxic. In another exemplary embodiment, ethylene glycol is used to not only disperse graphene but also to control viscosity. Ethylene glycol is also biocompatible, making it ideal for use in biosensor applications. In another exemplary embodiment, Propylene glycol is used as a dispersant and viscosity modifier, like ethylene glycol. Propylene glycol helps disperse graphene flakes due to its polar nature and ability to interact with the graphene surface, ensuring a stable dispersion. Propylene glycol increases the viscosity of the graphene ink, making it suitable for various application methods like screen printing, aerosol printing, and inkjet printing. Propylene glycol is also non-toxic, making it ideal for biomedical applications.

Hydrocarbons are another class of hydrophobic fluid used in the exemplary graphene ink to stabilize and disperse graphene flakes effectively. Hydrocarbons act as solvents in graphene inks by utilizing their nonpolar nature to effectively disperse hydrophobic graphene flakes. Hydrocarbons, being non-polar, can interact with the hydrophobic surfaces of graphene flakes, reducing van der Waals forces that cause aggregation. This interaction helps maintain a stable dispersion of graphene flakes within the solvent. In one exemplary embodiment, Toluene is used for its good solvating power and ability to maintain stable graphene dispersions. As an aromatic hydrocarbon with a moderate boiling point (110° C.), it can be used with a wide range of substrates. In another exemplary embodiment, Xylene is used to produce a uniform graphene layer. As an aromatic hydrocarbon with a moderate boiling point (148-144° C.), it can also be used with a wide range of substrates. In another exemplary embodiment, Hexane is used for its quick-drying capabilities. As an aliphatic hydrocarbon with a low boiling point (69° C.), it is used with a wide range of substrates and volume manufacturing processes. In another exemplary embodiment, Cyclohexane is used to control viscosity and provide a stable dispersion in nonpolar environments. As an aliphatic hydrocarbon with a low boiling point (81° C.), it can be used with a wide range of substrates. In another exemplary embodiment, Heptane is used for dispersing graphene. As an aliphatic hydrocarbon with a low boiling point (98° C.), it can be used with a wide range of substrates. In another exemplary embodiment, Octane is used for creating stable, non-polar graphene dispersions. As an aliphatic hydrocarbon with a moderate boiling point (125° C.), it can be used with a wide range of substrates. These hydrocarbons are chosen based on their ability to effectively disperse graphene flakes, control viscosity, and provide suitable evaporation rates for uniform graphene film formation.

Silicone oil is another class of hydrophobic fluid used in exemplary graphene ink to stabilize and disperse graphene flakes effectively. Silicone oils are used as solvents in graphene inks due to their unique properties, including thermal stability, chemical inertness, and low surface tension. Silicone oils used in exemplary graphene ink dispersions are Polydimethylsiloxane (PDMS), Phenylmethylsiloxane, and Trimethylsiloxy-terminated Dimethylsiloxane. PDMS is used for its low viscosity, high thermal stability, and chemical inertness. Phenylmethylsiloxane is used for its good thermal stability and wetting properties. Trimethylsiloxy-terminated Dimethylsiloxane is used for controlling viscosity and high chemical resistance. Octamethyltrisiloxane is used to lower surface tension, volatility, and acts as a carrier fluid. Methyl phenyl polysiloxane provides thermal stability, chemical resistance, and optical properties due to its unique methyl and phenyl side groups. 3-Glycidoxypropyl trimethoxysilane is used to form strong chemical bonds between organic and inorganic materials, enhancing mechanical and chemical properties.

Fluorinated fluid is another class of hydrophobic fluid used in exemplary graphene ink to stabilize and disperse graphene flakes effectively. Fluorinated fluids are used in exemplary graphene ink dispersions for their unique properties, including chemical inertness, thermal stability, and low surface tension. Fluorinated fluids used in the exemplary graphene ink dispersions are Perfluorooctane (PFO), Fluorinert Electronic Liquids (e.g., FC-40, FC-70), Perfluorodecalin, and Hexafluorobenzene.

Chlorinated fluid is another class of hydrophobic fluid used in exemplary graphene ink to stabilize and disperse graphene flakes effectively. Chlorinated fluids are used in exemplary graphene ink dispersions for their unique ability to dissolve and stabilize graphene flakes. Most chlorinated fluids also have low boiling points, making them suitable for use with a wide range of substrates. Chlorinated fluids used in the exemplary graphene ink dispersions are Chloroform (Trichloromethane), Dichloromethane (Methylene Chloride), Tetrachloroethylene (Perchloroethylene), and Carbon Tetrachloride.

Surfactant is another class of hydrophobic fluid used in exemplary graphene ink to stabilize and disperse graphene flakes effectively. Surfactants are often used in graphene ink dispersions to improve the stability and uniformity of the dispersions by preventing graphene flakes from aggregating. Surfactants used in the exemplary graphene ink dispersions are Sodium Dodecyl Sulfate (SDS), Triton X-100, Tween 80 (Polysorbate 80), Cetyltrimethylammonium Bromide (CTAB), Pluronic F127, and Sodium Cholate. SDS is an anionic surfactant effective in stabilizing graphene dispersions by providing electrostatic repulsion between graphene flakes. Triton X-100 is a nonionic surfactant used to improve dispersion stability without significantly affecting the electrical properties of graphene. Tween 80 is a nonionic surfactant used for its biocompatibility. CTAB is a cationic surfactant used to provide good dispersion stability through electrostatic stabilization. Pluronic F127 is a nonionic surfactant used to stabilize graphene dispersions, especially in biomedical applications. Sodium Cholate is a bile salt surfactant used for stabilizing graphene in aqueous dispersions.

Blends are another class of hydrophobic fluid used in exemplary graphene ink to stabilize and disperse graphene flakes effectively. Blends comprising a combination of various concentrations of at least two hydrophobic fluids of the exemplary embodiments.

The classes of polymer binders in the exemplary embodiments include cellulose derivatives, polyvinyl pyrrolidone (PVP), polyvinyl alcohol (PVA), polyethylene glycol (PEG), polyurethane (PU), polystyrene (PS), acrylic polymers, epoxy resins, and polyimides.

Cellulose derivatives come from natural cellulose, these polymers offer excellent film-forming properties, mechanical strength, and biodegradability. Exemplary cellulose derivatives include ethyl cellulose (EC), methylcellulose (MC), hydroxypropyl cellulose (HEC), carboxymethyl cellulose (CMC), nitrocellulose microcrystalline cellulose (MCC), and nitrocellulose.

Polyvinylpyrrolidone (PVP) has excellent solubility in water and many organic solvents, enabling good film-forming properties and adhesive qualities. PVP is particularly useful in ink formulations where biocompatibility is required, including biosensors. PVP has various molecular weight grades, including PVP K15, PVP K40, PVP K90, crosslinked PVP, functionalized PVP, and PVP blends. These various types of PVP act as a stabilizing agent by preventing the aggregation of particles, ensuring a stable, homogenous mixture that leads to consistent printing results.

Polyvinyl Alcohol (PVA) is a versatile and effective polymer binder in graphene inks, providing improved dispersion, film formation, viscosity control, and improved adhesion. Exemplary types of PVA are FHPVA, PHPVA, low molecular weight PVA, high molecular weight PVA, cross-linked PVA, modified PVA, and PVA blends.

Polyethylene glycol (PEG) is used to introduce functional groups that interact with other components of the ink and substrate. PEG is also used as a dispersant and stabilizer for improved film formation and adhesion. Exemplary types of PEG are low molecular weight, high molecular weight, functionalized PEG, biocompatible PEG, PEG blends, and PEG-ssDNA conjugates.

Polyurethane (PU) is used as a polymer binder in the exemplary embodiment. PU is a film-forming agent that creates a uniform and continuous film after printing and curing. Some stand-out properties of PU are its mechanical flexibility, durability, chemical resistance, and thermal stability. PU also offers suitable rheological properties due to its shear-thinning behavior. Exemplary types of PU include Thermoplastic Polyurethane (TPU), Waterborne Polyurethane (WPU), Polyurethane Acrylate (PUA), Thermosetting Polyurethane (TSU), and Polyurethane Elastomers (PUE).

Polystyrene (PS), in the exemplary embodiment, is used as a binder and film-forming agent, creating a uniform and cohesive film over the substrate after printing and curing. This encapsulation helps in maintaining the dispersion of graphene particles within the film, ensuring good adhesion to the substrate. PS also provides rigidity and mechanical strength to the printed films. Exemplary types of Polystyrene (PS) include general-purpose polystyrene (GPPS), high-impact polystyrene (HIPS), Polystyrene Butadiene styrene (SBS), and Polystyrene Sulfonate (PSS).

Acrylic Polymers, in the exemplary embodiment, are used to improve film formation, mechanical strength, adhesion, and flexibility. Exemplary types of Acrylic Polymers include Poly (methyl methacrylate) (PMMA), Acrylic copolymers, Acrylic Dispersions, Acrylic Emulsions, and Acrylic Urethane Hybrids.

Epoxy Resins, in the exemplary embodiment, are used to improve mechanical properties, adhesion, chemical resistance, and thermal stability. Epoxy resins act as binders in graphene inks and can be cured through thermal or UC processes. Exemplary types of Epoxy Resins include Bisphenol A Diglycidyl Ether (BADGE), Bisphenol F Diglycidyl Ether (BFDGE), Novolac Epoxy Resins, Cycloaliphatic Epoxy Resins, Glycidyl Amine Epoxy Resins, and Multifunctional Epoxy Resins.

The classes of additives in the exemplary embodiments are dispersing agents, rheology modifiers, surfactants, plasticizers, cross-linking agents, conductivity improvers, anti-foaming agents, UV stabilizers, flame retardants, adhesion promoters, and other types of additive solvents.

Dispersing agents are critical in the formulation of graphene inks as they help to stabilize the graphene particles, preventing agglomeration and ensuring uniform dispersion. The types of dispersing agents in the exemplary embodiment include polymers, small molecules, ionic liquids, biopolymers, and amphiphilic molecules.

Polymers ensure uniform dispersion and stability of graphene particles, preventing aggregation by providing steric stabilization and electrostatic stabilization. Types of polymers include surfactant polymers, cationic polymers, anionic polymers, amphiphilic polymers, and polyelectrolytes. Surfactant polymers can be nonionic, anionic, cationic, or amphoteric. Exemplary embodiments of nonionic surfactant polymers include polyethylene glycol (PEG) and pluronic block copolymers, which are triblock copolymers consisting of a central hydrophobic block of polypropylene glycol (PPG) flanked by two hydrophobic blocks of polyethylene glycol (PEG). Anionic surfactant polymers carry a negative charge, which makes them effective in preventing particle aggregation through electrostatic repulsion. Exemplary embodiments include polyacrylic acid (PAA) and poly (sodium 4-styrene sulfonate) (PSS). PAA can effectively stabilize suspensions through both steric and electrostatic mechanisms, and PSS is used to stabilize particles in aqueous dispersions due to its strong anionic character. Cationic surfactant polymers carry a positive charge, which makes them suitable for applications where interaction with negatively charged surfaces or particles is required. Polyethyleneimine (PEI) and Poly (diallyldimethylammonium chloride) (PDADMAC) are used for their strong electrostatic interactions. Amphoteric surfactant polymers carry both positive and negative charges depending on the pH of the solution. Proteins and polypeptides are natural polymers, like casein and synthetic polypeptides that act as amphoteric surfactants, including gelatin. Betaines are another example of an amphoteric surfactant polymer containing both quaternary ammonium and carboxylate groups. Polyelectrolytes are polymers with ionizable groups that dissociate in aqueous solutions, imparting charge to the polymer and enhancing its ability to stabilize dispersions through electrostatic interactions. Exemplary embodiments of polyelectrolytes include Poly (diallyldimethylammonium chloride) (PDADMAC), Polyethyleneimine (PEI), and Carboxymethyl Cellulose (CMC). Other exemplary polymer embodiments include water-soluble Polyvinylpyrrolidone (PVP), Polyethylene Glycol (PEG), Poly (sodium 4-styrene sulfonate) (PSS), Carboxymethyl Cellulose (CMC), Polyacrylic Acid (PAA), Polyvinyl Alcohol (PVA), and Hydroxypropyl Cellulose (HPC). Exemplary embodiments include water-insoluble Polystyrene (PS), Polyvinylidene Fluoride (PVDF), Poly (methyl methacrylate) (PMMA), Polyaniline (PANI), Polypyrrole (PPy), Polyethylene (PE), and Polypropylene (PP).

Small molecules are also used to effectively serve as dispersing agents in graphene inks by stabilizing the graphene sheets and preventing aggregation. Exemplary embodiments include N-Methyl-2-pyrrolidone (NMP), Dimethylformamide (DMF), Tetrahydrofuran (THF), 1,2-dichlorobenzene (DCB), Sodium Dodecyl Sulfate (SDS), Sodium Cholate, Ethyl Cellulose, Triton X-100, and Bovine Serum Albumin (BSA).

Ionic liquids (ILs) are salts in a liquid state at relatively low temperatures (often below 100° C.). They have unique properties including high thermal stability, low vapor pressure, and good solvating capabilities, making them effective dispersing agents for van Der Waal materials. Exemplary embodiments include 1-Butyl-4-methylimidazolium Hexafluorophosphate (BMIM-PF6), 1-Ethyl-4-methylimidazolium Ethyl Sulfate (EMIM-EtSO4), 1-Butyl-4-methylimidazolium Tetrafluoroborate (BMIM-BF4), 1-Ethyl-4-methylimidazolium Bis(trifluoromethyl sulfonyl)imide (EMIM-TFSI), 1-Butyl-4-methylimidazolium Chloride (BMIM-CI), 1-Butyl-4-methylimidazolium Dicyanamide (BMIM-DCA) and 1-Butyl-4-methylimidazolium Acetate (BMIM-Ac).

Biopolymers, due to their biocompatibility, biodegradability, and functional properties, are used as dispersing agents in van der Waals material ink. Exemplary embodiments include Chitosan, Alginate, Cellulose Derivatives, Gelatin, Pectin, Starch, and Polylactic Acid (PLA).

Amphiphilic molecules are compounds that contain both hydrophilic (water-loving) and hydrophobic (water-repelling) parts, which makes them effective dispersing agents for stabilizing graphene inks. These molecules can reduce surface tension and prevent the aggregation of graphene sheets. Exemplary embodiments of amphiphilic molecules used as dispersing agents in van der Waals material ink include Pluronic Block Copolymers, Triton X-100, Sodium Dodecyl Sulfate (SDS), Sodium Cholate, Octylphenol Ethoxylate (Igepal CA-640), Tween 80 (Polysorbate 80), and Sodium Dodecylbenzenesulfonate (SDBS).

Polyelectrolytes are polymers with ionizable groups that can dissociate in aqueous solutions, providing charge and enhancing the dispersion stability of graphene. Exemplary embodiments of polyelectrolytes used as dispersing agents in graphene inks are poly (sodium 4-styrene sulfonate) (PSS), Polyacrylic Acid (PAA), and Polyethyleneimine (PEI).

Rheology modifiers are essential components in the formulation of van der Waals material inks, as they play a role in adjusting the flow properties and stability of inks for specific printing techniques, including screen printing, inkjet printing, aerosol printing, pad printing, or gravure printing. They help maintain the stability of dispersions by preventing the sedimentation of particles and ensuring a uniform distribution of van der Waals material throughout the ink. Achieving a shear-thinning behavior, where the viscosity decreases with increasing shear rate, is essential for printing processes. Rheology modifiers enable this behavior, making the ink easier to spread and print while maintaining a higher viscosity at rest to prevent dripping or spreading. Properly adjusted rheology ensures that the ink can be printed uniformly without defects, including streaks, blotches, or uneven thicknesses. This is critical for applications requiring high precision and consistency, like flexible electronics. Rheology modifiers aid in the formation of smooth, defect-free films of van der Waal materials after printing, which is essential for the electrical and mechanical properties of the printed structures. Exemplary embodiments of rheology modifiers are thickeners, shear-thinning agents, viscoelastic modifiers, sol-gel rheology modifiers, and plasticizers.

Thickeners ensure proper viscosity and stability. Exemplary embodiments include Hydroxyethyl Cellulose (HEC), Xanthan Gum, Carboxymethyl Cellulose (CMC), Polyvinyl Alcohol (PVA), Ethyl Cellulose, Guar Gum, and Polyethylene Glycol (PEG).

Shear-thinning agents are used in printable inks to optimize their flow properties during the printing process. Shear-thinning agents decrease the viscosity of the ink under shear stress, including during the screen-printing process. This allows the ink to flow more easily through the screen mesh, resulting in smoother and more precise prints. Lower viscosity under shear enables the ink to penetrate fine mesh screens and reproduce intricate details and patterns more accurately. Shear-thinning inks regain their higher viscosity when the shear stress is removed, which prevents the ink from dripping, sagging, or spreading uncontrollably after printing. The controlled viscosity change helps in depositing uniform layers of ink, which is for applications like printed electronics, where layer thickness consistency affects performance. The shear-thinning behavior can improve the wetting properties of the ink, leading to better adhesion to the substrate and increased durability of the printed layers. By reducing viscosity under shear, shear-thinning agents help prevent the screen from clogging, which can occur if the ink is too thick. This ensures a more continuous and efficient printing process. Exemplary embodiments include Cellulose Derivatives, Polyvinyl Alcohol (PVA), Polyurethane Rheology Modifiers, Hydrophobically Modified Ethoxylated Urethane (HEUR), and Associative Thickeners.

Viscoelastic modifiers improve the balance between viscosity and elasticity, which are critical for the performance and quality of printed materials. Viscoelastic modifiers help control the flow properties of inks. They ensure that the ink spreads evenly on the substrate without excessive flow, which is essential for achieving sharp and well-defined prints. These modifiers help in forming uniform layers by preventing the ink from flowing too much during the printing process and by ensuring that it solidifies correctly after application. Viscoelastic modifiers can improve the adhesion of inks to various substrates, improving the durability of the printed material. They achieve this by optimizing the interaction between the ink and the substrate surface. These modifiers help control the drying and curing times of inks, ensuring that they dry at an appropriate rate. This prevents defects like cracking or uneven drying. By maintaining an optimal viscoelastic balance, these modifiers help prevent screen clogging during the printing process, which is critical for continuous and efficient printing. Viscoelastic modifiers contribute to the flexibility of printed materials, which is particularly important in applications like flexible electronics. Exemplary embodiments include Cellulose Derivatives, Polyurethane Rheology Modifiers, Polyvinyl Alcohol (PVA), Xanthan Gum, Hydrophilically Modified Ethoxylated Urethane (HEUR), Amorphous Polymers, Semicrystalline Polymers, Biopolymers, and Bitumen materials.

Sol-gel rheology modifiers control flow properties and improve stability in van der Waal material inks. The sol-gel process involves the transition of a solution into a gel, which provides unique rheological characteristics beneficial for printable inks. Sol-gel modifiers can help in the uniform dispersion of particles within the ink. The sol-gel network can entrap particles, preventing agglomeration and settling, thus maintaining a stable dispersion. Sol-gel systems can adjust the viscosity of inks, providing shear-thinning properties. Sol-gel rheology modifiers improve the printability of graphene inks by providing better control over ink flow and film formation. They help create smooth, uniform films upon drying, which is critical for the performance of printed electronic components. The sol-gel network can improve the mechanical properties of the printed films, making them more robust and durable. This is particularly beneficial for applications requiring flexible or stretchable electronics. Sol-gel rheology modifiers can be used to fine-tune the electrical properties of graphene inks. By controlling the dispersion and film formation, sol-gel systems can optimize the conductive pathways in the printed films. Exemplary embodiments include Silica-Based Sol-Gel Systems, Titania (TiO2) Sol-Gel Systems, Alumina (Al2O4) Sol-Gel Systems, Zirconia (ZrO2) Sol-Gel Systems, Hybrid Organic-Inorganic Sol-Gel Systems, Cerium Oxide (CeO2) Sol-Gel Systems, Iron Oxide (Fe2O4) Sol-Gel Systems, and Tungsten Oxide (WO4) Sol-Gel Systems.

Surfactants play a role in the formulation of graphene inks by improving dispersion stability, enhancing wetting properties, and preventing aggregation of graphene particles. Surfactants reduce the surface tension between graphene particles and the solvent, helping to disperse the particles uniformly throughout the ink. This prevents aggregation and sedimentation. Surfactants improve the wetting properties of the ink, ensuring better adhesion and spreading on various substrates. This is critical for achieving uniform coatings and high-quality prints. Exemplary embodiment types of surfactants include Anionic Surfactants, Cationic Surfactant, Nonionic Surfactant, Amphoteric (Zwitterionic) Surfactants, Flurosurfactant, and Silicone Surfactant.

Anionic surfactants improve dispersion, stability, and performance. Exemplary embodiments include Sodium, Dodecyl Sulfate (SDS), Sodium Lauryl Sulfate (SLS), Sodium Lauryl Ether Sulfate (SLES), Alkyl Benzene Sulfonates, and Sodium Stearate, Sodium Polyacrylate.

Cationic surfactants provide excellent wetting, emulsifying, and antimicrobial properties. Exemplary embodiments include Cetyltrimethylammonium Bromide (CTAB), Benzalkonium Chloride, Tetradecyltrimethylammonium Bromide (TTAB), Dodecyltrimethylammonium Bromide (DTAB), Laurylamine Hydrochloride, and Stearyldimethylbenzyl Ammonium Chloride.

Nonionic surfactants do not ionize in aqueous solutions and typically contain hydrophilic (water-loving) groups that are not charged, including hydroxyl or ether groups. Exemplary embodiments include Polysorbates (e.g. Tween 20, Tween 40, Tween 60, Tween 80), Sorbitan Esters (Span 20, Span 40, Span 60, Span 80), Alkyl Phenol Ethoxylates (e.g. Nonylphenol ethoxylates (NP-10), Octylphenol ethoxylates (Triton X-100)), Alcohol Ethoxylates (e.g. Lauryl Alcohol Ethoxylate, Cetyl Alcohol Ethoxylate, Stearyl Alcohol Ethoxylate), Fatty Acid Ethoxylates (e.g. Stearic Acid Ethoxylate, Oleic Acid Ethoxylate), Amine Ethoxylates (e.g. Tallow Amine Ethoxylate, Cocoamine Ethoxylate), Polyoxyethylene Alkyl Ethers (e.g. Brij 40, Brij 45, Brij 58), Polyethylene Glycol Esters (e.g. PEG 400 Monostearate, PEG 400 Dioleate).

Amphoteric surfactants contain both acidic and basic groups and can act as either anionic or cationic surfactants depending on the pH of the solution. Exemplary embodiments include Cocamidopropyl Betaine (CAPB), Cocamidopropyl Hydroxysultaine (CAHS), Lauryl Betaine, Sodium Lauriminodipropionate, Disodium Cocoamphodiacetate, Disodium Laureth Sulfosuccinate and Sodium Cocoamphoacetate.

Fluorosurfactants contain fluorine atoms in their structure, making them highly effective in reducing surface tension even at very low concentrations. They are known for their exceptional chemical and thermal stability, and resistance to harsh conditions. Exemplary embodiments include: Perfluorooctanoic Acid (PFOA), Perfluorooctanesulfonic Acid (PFOS), Zonyl FSN Fluorosurfactant, Capstone FS-40 Fluorosurfactant, Novec Fluorosurfactants, Fluorad FC-4440, and Forafac Fluorosurfactants.

Silicone surfactants, also known as organosilicon surfactants, are a class of surfactants where the hydrophobic part of the molecule is a silicone-based chain. These surfactants are widely used in various industries due to their unique properties, including excellent spreading, wetting, and foam control capabilities. Exemplary embodiments include Dimethicone Copolyol, Polyether Modified Siloxanes (e.g., Silwet L-77), Trisiloxane Surfactants (e.g., Trisiloxane ethoxylates like Silwet 408, Silsoft 410), Amine Functional Silicones (e.g., Amodimethicone), Silicone Polyethers (e.g., PEG-12 Dimethicone), Silicone Quaternary Ammonium Compounds (e.g., Quaternium-80), and Silicone Glycols (e.g., Bis-PEG/PPG-14/14 Dimethicone).

Plasticizers improve ink flexibility, durability, and workability. Plasticizers reduce the brittleness of the dried film. They intercalate between polymer chains, increasing their mobility and flexibility. By increasing the flexibility of the ink film, plasticizers help prevent cracking and flaking, enhancing the durability of the printed material. Plasticizers can modify the viscosity of inks, making them easier to process and apply. This is particularly important for achieving the desired flow properties in various printing techniques. Plasticizers can improve the adhesion of inks to various substrates by increasing the ink's ability to form a continuous film on the substrate surface. The addition of plasticizers can lead to smoother ink films, which improves the overall print quality. This includes better color uniformity and reduced defects, including pinholes and orange peel effects. Some plasticizers can also act as coalescing agents, aiding in the film formation process and reducing the drying time of the ink. Exemplary embodiments include Phthalate Esters (e.g. Diethyl Phthalate (DEP), Dibutyl Phthalate (DBP), Di(2-ethylhexyl) Phthalate (DEHP), Di(2-ethylhexyl) phthalate (DEHP), Diisononyl phthalate (DINP), Diisodecyl phthalate (DIDP)), Adipate Esters (e.g. Di(2-ethylhexyl) Adipate (DEHA), Diisodecyl Adipate (DIDA), Diisononyl adipate (DINA)), Trimellitates (e.g. Tris(2-ethylhexyl) trimellitate (TOTM), Triisononyl trimellitate (TINTM)), Epoxy Esters (e.g. Epoxidized soybean oil (ESO), Epoxidized linseed oil (ELO)), Phosphate Esters (e.g. Tris(2-butoxyethyl) phosphate (TBEP), Triphenyl phosphate (TPP)), Citrate Esters (e.g. Triethyl Citrate (TEC), Acetyl Tributyl Citrate (ATBC)), Sebacate Esters (e.g. Dioctyl Sebacate (DOS), Diisodecyl Sebacate (DIDS)), Glycol Ethers (e.g. Propylene Glycol (PG), Polyethylene Glycol (PEG), Dipropylene Glycol (DPG), Epoxidized Soybean Oil (ESO)) and Benzoate Esters (e.g. Diethylene Glycol Dibenzoate (DEGDB), and Dipropylene Glycol Dibenzoate (DPGDB)).

Cross-linking agents improve the mechanical properties, chemical resistance, and durability of the final printed films. These agents work by forming chemical bonds between polymer chains, resulting in a more robust network.

Epoxy-Based Cross-Linking Agents provide excellent adhesion, chemical resistance, and mechanical strength. Exemplary embodiments include Diglycidyl Ether of Bisphenol A (DGEBA), Epoxy Novolac Resins (e.g. Phenol Novolac epoxy (PNE), Cresol Novolac epoxy (CNE)), Glycidyl Methacrylate (GMA), Epoxidized Soybean Oil (ESO), Epoxy-Functional Silanes (e.g. (4-Glycidyloxypropyl) trimethoxysilane (GPTMS)), Cycloaliphatic Epoxy Resins (e.g. 4,4-Epoxycyclohexylmethyl-4,4-epoxycyclohexane carboxylate (ERL-4221)) and Aliphatic Epoxy Resins (e.g. 1,4-Butanediol diglycidyl ether (BDGE).

Amino cross-linking agents improve the durability, chemical resistance, and mechanical properties of inks. These agents work by reacting with functional groups in the ink formulation to form strong covalent bonds. Exemplary embodiments include Melamine-Formaldehyde (MF) Resins, Urea-Formaldehyde (UF) Resins, Benzoguanamine-Formaldehyde (BGF) Resins, Hexamethoxymethylmelamine (HMMM), Glycoluril-Based Cross-Linkers (e.g., Tetra(methoxymethyl) glycoluril), and Melamine-Urea-Formaldehyde (MUF) Resins.

Isocyanate cross-linking agents are widely used in ink formulations to improve durability, chemical resistance, and mechanical properties. These agents react with hydroxyl, carboxyl, and amine groups in polymers to form strong urethane or urea linkages. Exemplary embodiments include Hexamethylene Diisocyanate (HDI), Toluene Diisocyanate (TDI), Isophorone Diisocyanate (IPDI), Methylene Diphenyl Diisocyanate (MDI), Polyisocyanates (e.g., Desmodur® N Series), Polymeric Isocyanates, and Blocked Isocyanates.

Aziridine cross-linking agents improve adhesion, chemical resistance, and mechanical properties. These agents react with functional groups, including carboxyl, hydroxyl, and amine groups, to form strong covalent bonds. Exemplary embodiments include Tris(2-methyl-1-aziridinyl) phosphine oxide (MAPO), Trimethylolpropane Tris(2-methyl-1-aziridine) propionate (TTA), Pentaerythritol Tris(2-methyl-1-aziridine) propionate (PTA), Polyfunctional Aziridine (PFAZ), and CycloaliphaticAziridines.

Acrylate and methacrylate cross-linking agents improve properties including adhesion, flexibility, and chemical resistance. These agents contain acrylate or methacrylate groups that can polymerize to form cross-linked networks. Exemplary embodiments include Trimethylolpropane Triacrylate (TMPTA), Pentaerythritol Triacrylate (PETA), Ethoxylated Trimethylolpropane Triacrylate (ETMPTA), Hexanediol Diacrylate (HDDA), Diethylene Glycol Diacrylate (DEGDA), 1,6-Hexanediol Dimethacrylate (HDDMA), Triethylene Glycol Dimethacrylate (TEGDMA), Urethane Acrylates, and Bisphenol A Diglycidyl Ether Dimethacrylate (Bis-GMA).

Silane cross-linking agents improve adhesion, chemical resistance, and mechanical properties by forming covalent bonds with the substrate and the ink components. There are functional silanes such as epoxy, amino, vinyl methacryloxy, and mercapto, and alkoxysilanes such as monoalkoxysilanes. Exemplary embodiments include Vinyltrimethoxysilane (VTMS), (4-Glycidyloxypropyl) trimethoxysilane (GPTMS), (4-Methacryloxypropyl) trimethoxysilane (MPTMS), tetramethoxysilane (TMOS), (4-Aminopropyl) triethoxysilane (APTES), Phenyltrimethoxysilane (PTMS), Vinyltriethoxysilane (VTES), and Bis (trimethoxysilylpropyl) amine (BTMSPA).

Phenolic resins provide excellent thermal stability, chemical resistance, and mechanical properties. Exemplary embodiments include Novolac Resins, Resol Resins, Modified Phenolic Resins, Alkyl Phenolic Resins, Polyphenolic Resins, and Cresol-Formaldehyde Resins.

Conductivity improvers are used to improve the electrical properties of inks. The exemplary embodiment classes of conductivity improvers include Metallic Nanoparticles, Carbon-Based Materials, Conductive Polymers, Metal Oxides & Compounds, Organometallic Compounds, Hybrid Materials, Ionic Liquids, and Doped Materials.

Metallic Nanoparticles are known for their high electrical conductivity and stability. Exemplary embodiments include silver nanoparticles, gold nanoparticles, copper nanoparticles, and nickel nanoparticles. Silver offers superior conductivity, while gold resists oxidation. Copper is cost-effective compared to gold and silver, although prone to oxidation, and thus is often used with protective coatings. Nickel nanoparticles are not only conductive but also compatible with other materials.

Carbon-based materials, also known for their high electrical properties, are less stable than metallic nanoparticles. Exemplary embodiments include graphene, carbon nanotubes (CNTs), carbon black, graphene oxide (GO), and reduced graphene oxide (rGO). Graphene offers excellent electrical, thermal, and mechanical properties, while CNTs offer comparable conductivity and mechanical strength. Carbon black, though less conductive than graphene and CNTs, is more cost-effective.

Conductive Polymers are blended with other materials to improve conductivity. Exemplary embodiments include (Poly(4,4-ethylenedioxythiophene) polystyrene sulfonate) (PEDOT), Polyaniline, and Polyprrrole.

Metal Oxides & Compounds provide conductive properties while being transparent. Exemplary embodiments include Indium Tin Oxide (ITO) and Zinc Oxide (ZnO).

Organometallic compounds provide high conductivity, and flexibility and are used in transparent films. Exemplary embodiments include silver nanowires and gold nanowires.

Hybrid Materials combine the properties of conductivity-enhancing materials to achieve desired properties. Exemplary embodiments include Graphene-CNT Hybrids, Metal-Polymer Composites, Graphene-Metal Nanoparticle Hybrids, and Graphene-Conductive Polymer Hybrids (e.g., Graphene-PEDOT: PSS).

Ionic Liquids improve dispersion and improve conductivity. Exemplary embodiments include Imidazolium-Based Ionic Liquids and Pyridinium-Based Ionic Liquids.

Doped Materials improve the conductivity of inks for specialized applications, including conductive sensor films. Exemplary embodiments include Boron-Doped Diamond and Nitrogen-Doped Graphene

Antifoaming agents or defoamers prevent the formation of foam during manufacturing and application processes. Foam can cause defects in the ink film and interfere with the printing process. Key considerations for choosing an anti-foaming agent include stability, performance, and regulatory compliance. The exemplary classes of anti-foaming agents are Silicone-Based Agents, Non-Silicone Organic Agents, Particulate Agents, Polymer-Based Agents, Surfactant-Based Agents, and Combination-Based Agents.

Silicone-based agents reduce and prevent foam formation. Exemplary embodiments include Polydimethylsiloxane (PDMS), Silicone Emulsions, Modified Silicones, Silicone-Glycol Copolymers, Hydrophobic Silica-treated Silicones, Fluorosilicones, Silicone Antifoam Powders, Fumed Silica, and Silicone Antifoam Compounds. PDMS has high spreading capability on foam surfaces, is effective at low concentrations, and is chemically inert and stable over a wide range of temperatures. PDMS's low surface tension helps break foam bubbles. Silicone Emulsions are dispersed in water, making them easy to incorporate into water-based inks and provide immediate foam control and long-term stability. They are typically non-ionic, ensuring compatibility with a variety of formations. Modified Silicones are silicones modified with organic functional groups (e.g., alkyl or aryl groups) to improve compatibility with organic solvents and other ink components. Silicone-glycol copolymers improve dispersibility in aqueous systems, enhancing compatibility with both aqueous and non-aqueous formulations. Hydrophobic Silica-treated Silicones consist of silica particles treated with silicone to improve defoaming properties. Fluorosilicones contain fluorine atoms that provide unique chemical properties, including lower surface tension than standard silicones and excellent resistance to chemical attack and stability under harsh conditions. Silicone Antifoaming Powders are dry powders formed of silicone antifoam agents that are combined with carriers like silica. Silicone Antifoam Compounds are concentrated silicone antifoam agents in a viscous or paste form that are highly effective in small doses.

Non-silicone organic Anti-foaming agents are used when silicone-based agents negatively impact the formulation or application. Exemplary embodiments include Mineral Oils, Vegetable Oils (Soybean oil, canola oil, and castor oil) Fatty Alcohols (e.g. stearyl alcohol and cetyl alcohol), Fatty Acids and Esters (e.g. oleic acid, stearic acid, and glycerol esters), Polypropylene Glycol (PPG), Alkylphenol Ethoxylates, EO/PO Copolymers (Ethylene Oxide/Propylene Oxide Copolymers), Organic Esters (e.g. Isopropyl myristate and methyl laurate, Phosphates and Phosphoric Acid Esters and Glycol Ethers (e.g. butyl glycol and hexyl glycol).

Particulate antifoaming agents break down and prevent foam by destabilizing foam bubbles through mechanical means. These agents are typically solid particles that interfere with the stability of foam, causing it to collapse. Exemplary embodiments include Hydrophobic Silica, Hydrophilic Silica, Wax Dispersions (e.g., micronized polyethylene wax, polypropylene wax, Talc, Magnesium Stearate, Calcium Carbonate, Zinc Stearate, Polymeric Beads (e.g., polystyrene beads and PMMA (polymethyl methacrylate) beads, Clay Particles (e.g., bentonite and kaolin), Perlite, and Diatomaceous Earth.

Polymer antifoaming agents control foam in various industrial applications, including inks. These agents work by reducing surface tension and disrupting the formation of foam bubbles. Exemplary embodiments include Polyacrylates, Polyethers (e.g., polyethylene glycol (PEG) and polypropylene glycol (PPG)), EO/PO Copolymers (Ethylene Oxide/Propylene Oxide Copolymers), Polyvinyl Alcohol (PVA), Polydimethylsiloxane-Polyether Copolymers, Polyurethanes, Polyacrylamides, Polycarboxylates, Polysiloxane-Modified Polyethers and Block Copolymers (e.g., Pluronics (PEO-PPO-PEO triblock copolymers).

Surfactant-based antifoaming agents are commonly used in inks to control foam by reducing surface tension and disrupting the stability of foam bubbles. Exemplary embodiments include Nonionic Surfactants (e.g. ethoxylated alcohols and alkylphenol ethoxylates), Anionic Surfactants (e.g. sodium lauryl sulfate (SLS), and dioctyl sulfosuccinate (DOSS)), Amphoteric Surfactants (e.g. cocamidopropyl betaine and alkyl betaines), Silicone-Polyether Copolymers, Alkylphenol Ethoxylates (e.g. nonylphenol ethoxylate and octyl phenol ethoxylate), EO/PO Copolymers (Ethylene Oxide/Propylene Oxide Copolymers), Fatty Acid Esters (e.g. Sorbitan esters (Span) and polysorbates (Tween)), Alkyl Polyglucosides (APGs) and Fluorosurfactants (e.g. perfluorooctanoic acid (PFOA) derivatives).

Combination defoaming agents blend different types of defoamers to achieve improved performance by leveraging the strengths of each component. These combinations can provide superior foam control, stability, and compatibility with various ink formulations. Exemplary embodiments include: Silicone and Non-Silicone Blends (e.g. Polydimethylsiloxane (PDMS) with mineral oils or fatty alcohols), Silicone and Organic Polymer Blends (e.g. PDMS with polyacrylates or polyethylene glycol (PEG)), Silicone and Surfactant Blends (e.g. Silicone oils with nonionic surfactants like ethoxylated alcohols or EO/PO copolymers), Silicone-Polyether Copolymer Blends (e.g. Copolymers combining silicone with polyether groups), Silica-Silicone Blends (e.g. Hydrophobic silica treated with silicone oils or PDMS), Organic Polymer and Surfactant Blends (e.g. Polyacrylates or polyurethanes combined with nonionic or anionic surfactants), Fatty Acid Ester and Mineral Oil Blends (e.g. Sorbitan esters (Span) or polysorbates (Tween) combined with mineral oils), Fluorosurfactant and Silicone Blends (e.g. Perfluorooctanoic acid (PFOA) derivatives combined with silicone oils) and Alkyl Polyglucoside and Silicone Blends (e.g. Alkyl polyglucosides (APGs) combined with silicone oils or PDMS).

UV stabilizers are additives used in conductive inks primarily to improve the durability and performance of the ink when exposed to ultraviolet (UV) light. UV stabilizers act as protective agents by absorbing or dissipating UV radiation. This prevents the UV light from degrading the organic components of the conductive ink. Many conductive inks contain organic materials that can degrade or change color when exposed to UV light over time. By protecting against UV degradation, stabilizers can extend the lifespan of conductive ink once it is applied to a substrate. Exemplary types of UV Stabilizers include UV Absorbers (UVA), Hindered Amine Light Stabilizers (HALS), Quenchers, Antioxidants, and Other Additives. UV absorbers work by absorbing UV radiation and converting it into harmless heat, thus preventing the UV light from reaching and damaging the substrate or the ink components. HALS act as radical scavengers, inhibiting the degradation of polymers and other organic materials caused by UV exposure. They stabilize the ink by neutralizing free radicals that are generated during the UV degradation process. Quenchers quench the excited states of photoreactive species, thereby preventing the formation of free radicals and other reactive species that can lead to degradation. While primarily used for thermal oxidation stabilization, some antioxidants also provide limited UV stabilization by inhibiting free radical formation and chain reactions initiated by UV light. Other additives include light stabilizers, which combine properties of UV absorbers and HALS, providing broad-spectrum protection against UV radiation and oxidative degradation.

Flame Retardants improve the fire resistance of printed materials. The process involves selecting appropriate flame-retardant chemicals based on the ink's composition and the desired properties. Exemplary classes of flame retardant additives include Halogenated Flame Retardants, Phosphorus-Based Flame Retardants, Inorganic Flame Retardants, Nitrogen-Based Flame Retardants, and Intumescent Systems. Exemplary embodiments of Halogenated Flame Retardants include Brominated Flame Retardants (e.g., Decabromodiphenyl Ether (DecaBDE), Octabromodiphenyl Ether (OctaBDE), tetrabromobisphenol A (TBBPA), Hexabromocyclododecane (HBCD), Pentabromodiphenyl Ether (PentaBDE), Ethylene Bis(tetrabromophthalimide) (EBTBP), Tetrabromophthalic Anhydride (TBPA) and Bis(tribromophenoxy)ethane (BTBPE)), Chlorinated Flame Retardants (e.g. Chlorinated Paraffins, Dechlorane Plus, Tetrachlorophthalic Anhydride (TCPA), Tris(2-chloroethyl) Phosphate (TCEP) and Tris(1-chloro-2-propyl) Phosphate (TCPP)), Combination of Bromine and Chlorine (e.g., Polybrominated Biphenyls (PBBs), Polybrominated Diphenyl Ethers (PBDEs) and 1,2-Bis(2,4,6-tribromophenoxy)ethane (BTBPE)).

Adhesion promoters improve the bonding between the ink and the substrate. Different classes of adhesion promoters are used based on the type of ink, substrate, and desired performance characteristics. Exemplary classes of adhesion promoters include Silane Coupling Agents, Phosphate Esters, Organotitanates, Polyolefin-based Adhesion Promoters, Maleic Anhydride Grafted Polymers, Polyurethane Dispersions (PUDs), Epoxy Resins, Acrylic-based Adhesion Promoters, Polyvinyl Butyral (PVB), Ethylene Vinyl Acetate (EVA) Copolymers, and Adhesion Promoting Primers.

Silane coupling agents improve adhesion to various substrates. Exemplary embodiments include: Vinyl Silanes (e.g., vinyltrimethoxysilane (VTMS) and vinyltriethoxysilane (VTES), Epoxy Silanes (e.g., glycidoxypropyltrimethoxysilane (GPTMS) and glycidoxypropyltriethoxysilane (GPTES)), Amino Silanes (e.g. aminopropyltriethoxysilane (APTES) and aminopropyltrimethoxysilane (APTMS), N-(2-Aminoethyl)-4-aminopropyltrimethoxysilane (AEAPTMS), Methacryloxy Silanes (e.g. methacryloxypropyltrimethoxysilane (MPTS) and methacryloxypropyltriethoxysilane (MPTES)), Chloro Silanes (e.g., chloropropyltrimethoxysilane (CPTMS) and chloropropyltriethoxysilane (CPTES), Isocyanato Silanes (e.g. isocyanatopropyltriethoxysilane (IPTES) and isocyanatopropyltrimethoxysilane (IPTMS)), Mercapto Silanes (e.g., mercaptopropyltrimethoxysilane (MPTMS) and mercaptopropyltriethoxysilane (MPTES)), Alkyl Silanes (e.g., octyltriethoxysilane (OTES) and hexadecyltrimethoxysilane (HDTMS), Phenyl Silanes (e.g., phenyltrimethoxysilane (PTMS) and phenyltriethoxysilane (PTES)) and Thiocyano Silanes (e.g. Thiocyanatopropyltriethoxysilane (TCPTES) and Thiocyanatopropyltrimethoxysilane (TCPTMS)).

Phosphate esters are selected based on the type of ink, substrate, and the desired performance characteristics of the printed material. They work by forming strong bonds between the ink and the substrate, improving durability and adhesion. Exemplary embodiments include 2-Ethylhexyl Phosphate, Polyvinylphosphonic Acid (PVPA), Tris(2-butoxyethyl) Phosphate (TBEP), Dimethyl Phosphate (DMP), Diethyl Phosphate (DEP), Dibutyl Phosphate (DBP), Triphenyl Phosphate (TPP), Tricresyl Phosphate (TCP), Tris(2-chloroethyl) Phosphate (TCEP), Tris(1-chloro-2-propyl) Phosphate (TCPP), and Phosphoric Acid, Mono- and Di-Esters with Ethylene Glycol.

Oganotitanates improve bonding between the ink and various substrates. Exemplary embodiments include: Tetra(2-ethylhexyl)titanate (TOT), Diisopropyl bis(dioctyl phosphate)titanate (DIPDP), Titanium(IV) isopropoxide (TIPT or TTIP), Di(dioctyl pyrophosphate) ethyl titanate (DOPET), Titanium(IV) butoxide (TBT or TnBT), Titanium(IV) acetylacetonate (TAA), Triethanolamine Titanate (TEAT), Titanium(IV) di(2-ethylhexyl)phosphonate (DEHPT), Titanium(IV) bis(ethyl acetoacetate) diisopropoxide (ETPI) and Titanium(IV) ethoxide (TET).

Polyolefin-based adhesion promoters improve the adhesion of inks to polyolefin substrates, including polyethylene (PE) and polypropylene (PP). Exemplary embodiments include Chlorinated Polyolefins (CPOs) (e.g., Chlorinated Polypropylene (CPP) and Chlorinated Polyethylene (CPE)), MaleicAnhydride Grafted Polyolefins (e.g. Maleic Anhydride Grafted Polypropylene (MAH-PP) and Maleic Anhydride Grafted Polyethylene (MAH-PE)), Acrylic-Modified Polyolefins, Functionalized Polyolefins (e.g. Ethylene Acrylic Acid (EAA) Copolymers and Ethylene Methacrylic Acid (EMAA) Copolymers)), Polypropylene-Maleic Anhydride Copolymers, Ethylene-Vinyl Acetate (EVA) Copolymers, Terpolymers of Ethylene, Propylene, and a Diene (EPDM) and Polyolefin Elastomers (POEs).

Maleic anhydride grafted polymers are widely used as adhesion promoters due to their ability to improve bonding to a variety of substrates, particularly non-polar surfaces like polyolefins. Exemplary embodiments include Maleic Anhydride Grafted Polypropylene (MAH-PP), Maleic Anhydride Grafted Polyethylene (MAH-PE), Maleic Anhydride Grafted Ethylene Propylene Diene Monomer (MAH-EPDM), Maleic Anhydride Grafted Styrene-Butadiene-Styrene (MAH-SBS), Maleic Anhydride Grafted Ethylene Vinyl Acetate (MAH-EVA), Maleic Anhydride Grafted Polybutene-1 (MAH-PB), Maleic Anhydride Grafted Polyisobutylene (MAH-PIB), Maleic Anhydride Grafted Linear Low-Density Polyethylene (MAH-LLDPE), MaleicAnhydride Grafted High-Density Polyethylene (MAH-HDPE) and Maleic Anhydride Grafted Polyolefin Elastomers (MAH-POE).

Polyurethane adhesion promoters improve adhesion to various substrates, providing flexibility, durability, and chemical resistance. Exemplary embodiments include Polyurethane Dispersions (PUDs) (e.g., Aliphatic Polyurethane Dispersions and Aromatic Polyurethane Dispersions), Blocked Isocyanates (e.g., Caprolactam-blocked Isocyanates and MEKO-blocked Isocyanates), Polycarbonate Polyurethane (PCU), Polyester Polyurethane (PEU), Polyether Polyurethane (PETU), Polyurethane Prepolymers (e.g., NCO-terminated Prepolymers and OH-terminated Prepolymers), Waterborne Polyurethanes, UV-curable Polyurethanes, Acrylic-Polyurethane Hybrids, and Silicone-Modified Polyurethanes.

Epoxy resins are commonly used as adhesion promoters in inks due to their excellent bonding properties and chemical resistance. Exemplary embodiments include Bisphenol A Epoxy Resins (e.g., Bisphenol A Diglycidyl Ether (BADGE) and Epoxy Novolac Resins), Bisphenol F Epoxy Resins (e.g., Bisphenol F Diglycidyl Ether (BFDGE)), Novolac Epoxy Resins, Cycloaliphatic Epoxy Resins (e.g., Cyclohexanedimethanol Diglycidyl Ether (CHDGE)), Glycidylamine Epoxy Resins (e.g., Tetraglycidyl Methylenedianiline (TGDDM)), Flexible Epoxy Resins (e.g. Polypropylene Glycol Diglycidyl Ether (PPGDGE)), Phenolic Epoxy Resins, Halogenated Epoxy Resins, Waterborne Epoxy Resins (e.g., Emulsified Bisphenol A Epoxy Resins and Modified Epoxy Emulsions), Modified Epoxy Resins (e.g., Rubber-Modified Epoxies, Acrylic-Modified Epoxies and Silicone-Modified Epoxies) and Epoxy Esters.

Acrylic-based adhesion promoters have excellent adhesion properties and provide flexibility and chemical resistance. Exemplary embodiments include Acrylic Acid (AA), Methacrylic Acid (MAA), Acrylic Esters (e.g., Methyl Acrylate (MA), Ethyl Acrylate (EA), Butyl Acrylate (BA) and 2-Ethylhexyl Acrylate (2-EHA)), Methacrylic Esters (e.g., Methyl Methacrylate (MMA), Ethyl Methacrylate (EMA), Butyl Methacrylate (BMA) and 2-Ethylhexyl Methacrylate (2-EHMA)), Hydroxyethyl Acrylate (HEA), Hydroxyethyl Methacrylate (HEMA), Glycidyl Methacrylate (GMA), Acrylic Copolymers (e.g. Styrene-Acrylic Copolymers, Acrylic-Polyurethane Hybrids, and Acrylic-Silicone Hybrids), Acrylic Oligomers, Acrylic Dispersions, Acrylic Emulsions (e.g., Vinyl Acrylic Emulsions and Pure Acrylic Emulsions), Adhesion Promoting Monomers (e.g., N-(2-Aminoethyl) Methacrylate (AEMA) and Acrylic Acid Grafted Polymers) and Acrylic Adhesion Primers.

Polyvinyl Butyral is used in inks and coatings for its strong bonding capabilities, flexibility, and clarity. Exemplary embodiments include Standard Polyvinyl Butyral (PVB) Resins (e.g., PVB-16 and PVB-40), Modified Polyvinyl Butyral (PVB) Resins (e.g., Plasticized PVB and UV-resistant PVB), PVB with Functional Groups (e.g., Carboxylated PVB and Hydroxylated PVB), Blended PVB Formulations (PVB-Acrylic Blends and PVB-Polyurethane Blends), High-Performance PVB Grades (PVB for High-Temperature Applications and PVB for Chemical Resistance), and PVB with Additives (e.g., PVB with Silane Coupling Agents and PVB with Phosphate Esters).

Ethylene Vinyl Acetate (EVA) copolymers provide excellent flexibility, adhesion properties, and compatibility with a variety of substrates. Exemplary embodiments include Standard EVA Copolymers (e.g., Low VA Content EVA, Medium VA Content EVA and High VA Content EVA), Functionalized EVA Copolymers (e.g., Acrylic Modified EVA and Maleic Anhydride Grafted EVA (MAH-EVA)), Blended EVA Formulations (e.g. EVA-Polyethylene Blends, EVA-Polypropylene Blends, and EVA-Polyurethane Blends), EVA Copolymers with Additives (e.g., EVA with Silane Coupling Agents, EVA with Phosphate Esters and EVA with UV Stabilizers), Specialty EVA Grades (e.g., High Melt Flow Index (MFI) EVA and Cross-linkable EVA), and EVA Copolymers for Specific Applications (e.g. EVA for Flexible Packaging Inks and EVA for Textile Inks).

Adhesion primers are specialized agents used in inks to improve the bonding of the ink to various substrates. They are often formulated with a combination of functional chemicals that provide strong interfacial bonding. Exemplary embodiments include Silane Coupling Agents, Phosphate Esters, Organotitanates, Polyolefin-Based Adhesion Promoters, Maleic Anhydride Grafted Polymers, Polyurethane-Based Adhesion Promoters, Epoxy Resins, Acrylic-Based Adhesion Promoters, Polyvinyl Butyral (PVB) Resins, Ethylene Vinyl Acetate (EVA) Copolymers, Functionalized Polymers and Copolymers, and Specialty Adhesion Primers.

Various categories represent different types of graphene materials, solvents or hydrophobic fluids, cellulose binders, and additives are used to improve the ink's properties. Cellulose derivatives are used in graphene inks to improve the ink's stability, viscosity, and compatibility with different substrates. Cellulose derivatives, including carboxymethyl cellulose (CMC) and hydroxypropyl cellulose (HPC), can stabilize graphene sheets in suspension. This prevents the graphene sheets from aggregating and ensures a uniform dispersion in the ink formulation. These derivatives can adjust the viscosity of the ink to suit different printing techniques, including inkjet, screen, or flexographic printing, etc. Cellulose derivatives can improve the adhesion of graphene to various substrates. This is particularly important for printed electronics, where strong adhesion ensures the durability and performance of the printed patterns. Cellulose Derivatives aid in forming a smooth and continuous film of graphene on the substrate. This is essential for creating conductive paths with minimal resistance and defects. Cellulose derivatives are compatible with various solvents used in ink formulations. This versatility allows for the development of water-based, solvent-based, or hybrid graphene inks.

Exemplary embodiments of oxidized graphene include Plasma Produced Graphene, Reduced Graphene Oxide (rGO), Graphene (GO), Holey Graphene, Single Layer Graphene, Multi-Layer Graphene, and Functionalized Graphene.

Exemplary embodiments of solvents or hydrophobic fluids include Alcohols, Glycols, Terpenes, Terpene Alcohol, Nonpolar Solvents, Organic Solvents, Aromatic Hydrocarbons, Aliphatic Hydrocarbons, and Surfactants.

Exemplary embodiments of cellulose binders include Ethyl Cellulose (EC), Methyl Cellulose (MC), Hydroxyethyl Cellulose (HEC), Hydroxypropyl Cellulose (HPC), Carboxymethyl Cellulose (CMC), Nitrocellulose, Microcrystalline Cellulose (MCC), and Nanocellulose.

Exemplary embodiments of additives include Dispersing Agents, Rheology Modifiers, Surfactants or Wetting Agents, Plasticizers, Cross-Linking Agents, Conductivity Improvers, Antifoaming Agents, UV Stabilizers, Flame Retardants, and Adhesion Promoters.

Ethyl cellulose has excellent film-forming properties, allowing it to form a continuous and uniform film when deposited onto a substrate. This property is essential for creating well-defined patterns or coatings of graphene inks during printing processes. Ethyl cellulose is soluble in a wide range of organic solvents, including alcohols, glycols, terpenes, terpene alcohols, nonpolar solvents, ketones, esters, hydrocarbons, and surfactants. This solubility allows for easy preparation of graphene ink formulations by dissolving ethyl cellulose and graphene flakes in a suitable solvent system. Ethyl cellulose acts as an effective binder for particulate materials, including graphene flakes, by forming a matrix that holds the graphene particles together. This binder-particle interaction improves the mechanical strength, adhesion, and stability of the printed or coated graphene films. Ethyl cellulose can modify the rheological properties of graphene inks, including viscosity and flow behavior. By adjusting the concentration of ethyl cellulose or using different grades with varying viscosities, the ink's rheology can be tailored to optimize printability and achieve desired ink flow characteristics. Ethyl cellulose is compatible with a wide range of ink components, including dispersants, surfactants, solvents, wetting agents, and additives. This compatibility allows for the incorporation of additional functionalities or performance-enhancing agents into graphene inks without adversely affecting the ink formulation or properties. The type of ethyl cellulose binder depends on various factors, including viscosity, solubility, dispersibility, printability, adhesion, and other film formation characteristics.

Low-viscosity ethyl cellulose grades are characterized by their relatively low molecular weight and viscosity. Typically, low-viscosity grades of ethyl cellulose have molecular weights ranging from approximately 10,000 g/mol to 40,000 g/mol and can vary depending on supplier specifications. Low-viscosity ethyl cellulose grades are suitable for applications where fast drying times and smooth film formation are desired.

Medium viscosity ethyl cellulose grades have moderate molecular weight and viscosity, providing a balance between flow properties and film-forming characteristics. They offer improved mechanical strength and adhesion compared to low-viscosity grades. Typically, medium viscosity grades of ethyl cellulose have molecular weights ranging from approximately 40,000 g/mol to 100,000 g/mol and can vary depending on supplier specifications.

High-viscosity ethyl cellulose grades have a higher molecular weight and viscosity, resulting in thicker and more viscous ink formulations. They are suitable for applications where higher ink viscosity is required to achieve better control over ink deposition and film thickness. High-viscosity ethyl cellulose grades can provide improved mechanical stability and durability to the printed or coated graphene films. Typically, high-viscosity grades of ethyl cellulose have molecular weights ranging from approximately 100,000 g/mol to 1,000,000 g/mol or even higher and can vary depending on supplier specifications.

Modified ethyl cellulose grades may include chemically modified or functionalized derivatives of ethyl cellulose with improved properties, including improved solubility, adhesion, or compatibility with specific ink components. These modified grades offer tailored performance characteristics to meet the requirements of specific graphene ink applications. Such examples of modified ethyl cellulose include, but are not limited to, Ethyl cellulose acetate (ECA), Ethyl hydroxyethyl cellulose (EHEC), Ethyl cellulose acetate propionate (ECAP), and Ethyl Cellulose Succinate (ECS). Ethyl cellulose acetate (ECA) is a derivative of ethyl cellulose where a portion of the hydroxyl groups on the cellulose backbone are replaced with acetate groups. This modification alters the solubility characteristics of ethyl cellulose, making it more compatible with certain solvents and improving its film-forming properties. Ethyl hydroxyethyl cellulose (EHEC) is a derivative of ethyl cellulose where ethyl groups along the cellulose backbone are partially replaced with hydroxyethyl groups. This modification increases the water solubility of ethyl cellulose, making it suitable for use in water-based formulations. Ethyl cellulose acetate propionate (ECAP) is a terpolymer composed of ethyl cellulose, cellulose acetate, and cellulose propionate. This combination of polymers offers a balance of properties, including solubility, film formation, and adhesion. Ethyl cellulose succinate (ECS) is a derivative of ethyl cellulose where succinate groups are attached to the cellulose backbone. This modification can improve the water solubility of ethyl cellulose and improve its compatibility with certain solvents.

Ethyl cellulose microspheres are spherical particles of ethyl cellulose with controlled particle size and morphology. These microspheres can be incorporated into inks to modify rheological properties, control viscosity, and improve ink stability and printability. In some applications, ethyl cellulose microspheres can be designed to encapsulate active ingredients or functional additives, including conductive nanoparticles or dopants. These microspheres can serve as reservoirs for the controlled release of these additives during printing or subsequent processing steps, allowing for precise control over the composition and properties of the printed graphene patterns. Ethyl cellulose microspheres can also be engineered to exhibit specific properties, including size, morphology, surface chemistry, and porosity. By adjusting these parameters, the properties of the microspheres can be tailored to meet the requirements of different ink formulations and printing processes, optimizing ink performance and printed graphene characteristics.

Ethyl cellulose nanoparticles are nanoscale particles of ethyl cellulose with high surface area and colloidal stability. These nanoparticles can be used as additives in inks to improve dispersion stability, control rheological properties, and improve ink performance during printing or coating processes.

Ethyl cellulose blends are when ethyl cellulose is combined with other materials to achieve specific properties or functionalities. Blending ethyl cellulose with polymers, particularly biocompatible polymers including polyvinylpyrrolidone (PVP), polyethylene glycol (PEG), or polyvinyl alcohol (PVA), can improve the biocompatibility, stability, and adhesion of graphene inks in biosensor applications. These blends help ensure proper immobilization of biomolecules (including antibodies, enzymes, or DNA probes) on the graphene surface while maintaining their bioactivity. In other exemplary embodiments, conductive polymers are blended with ethyl cellulose to improve the electrical conductivity of the ink and printed films. Such conductive polymers include, but are not limited to: Poly(4,4-ethylenedioxythiophene) Polystyrene Sulfonate (PEDOT: PSS), Polypyrrole (PPy), Polyaniline (PANI), Polythiophene (PTh), Poly(4-hexylthiophene) (P4HT), among other conductive polymers. In certain embodiments, these conductive polymers, as well as others, are incorporated into graphene inks either as dopants, modifiers, or blends to improve the electrical conductivity, electronic performance, and processability of the ink.

Polyvinylpyrrolidone (PVP) is a versatile polymer that improves dispersion stability, prevents agglomeration of graphene flakes, and improves the adhesion of graphene to substrates. Different types of PVP are used in exemplary embodiments, depending on factors including molecular weight, chemical structure, and rheological characteristics.

PVP K15 (Polyvinylpyrrolidone K15) is a low molecular weight grade of PVP, typically with a molecular weight range of 10,000 to 15,000 g/mol. In exemplary embodiments, it is used to improve dispersion stability and facilitate the exfoliation of graphene flakes due to its good solubility in water and organic solvents.

PVP K40 (Polyvinylpyrrolidone K40) is a medium molecular weight grade of PVP, with a molecular weight range of approximately 40,000 g/mol. In the exemplary embodiment, it is used as a dispersing agent to improve ink stability, prevent particle agglomeration, and improve the printability of the ink.

PVP K90 (Polyvinylpyrrolidone K90) is a high molecular weight grade of PVP, typically with a molecular weight range of 460,000 to 440,000 g/mol. In the exemplary embodiment, it is used to provide stronger binding and better adhesion between graphene flakes and substrates, leading to improved mechanical properties and film formation.

Crosslinked PVP, also known as PVP/VA (polyvinylpyrrolidone/vinyl acetate) copolymer, is a modified form of PVP where the polymer chains are crosslinked to improve stability and performance. In the exemplary embodiment, crosslinked PVP is used to improve ink stability, prevent sedimentation, and improve film formation on substrates.

Functionalized PVP are PVP derivatives that have been chemically modified to introduce specific functional groups or properties. These functionalized PVPs can be tailored to provide improved dispersibility, compatibility with different solvents, or specific interactions with graphene surfaces in ink formulations.

PVP Blends are when PVP is combined with other polymers, including polyethylene glycol (PEG), polyvinyl alcohol (PVA), or cellulose derivatives, to achieve synergistic effects and optimize ink performance for specific applications. In an exemplary embodiment, PVP is combined with polysaccharides. Blending PVP with polysaccharides, including but not limited to, hyaluronic acid, chitosan, or alginate, can improve the biocompatibility and adhesion of graphene inks in biosensor applications. These blends provide a biocompatible matrix for immobilizing biomolecules (e.g., aptamers, antibodies, enzymes, or DNA probes) on the graphene surface while maintaining their bioactivity and stability. In other embodiments, mixing PVP with conductive polymers, including poly(4,4-ethylenedioxythiophene) (PEDOT), polypyrrole (PPy), or polyaniline (PANI), can improve the electrical conductivity and electronic performance of graphene inks. These blends enable the fabrication of highly sensitive and selective biosensors for detecting target analytes in biological samples. In other embodiments, incorporating nanoparticles, including gold nanoparticles, silver nanoparticles, or magnetic nanoparticles into PVP blends can improve the sensitivity, specificity, and signal transduction of graphene-based biosensors. These blends facilitate the immobilization of biomolecules and improve the detection of target analytes through various sensing mechanisms, including surface plasmon resonance, electronic impedance spectroscopy, or surface-improved Raman scattering. In other embodiments, adding functional additives, including crosslinkers, redox mediators, or biocompatible surfactants to PVP blends, improves the stability, sensitivity, and reproducibility of graphene-based biosensors. These blends help optimize the sensor performance by improving biomolecule immobilization, minimizing non-specific binding, and reducing background noise in biosensing assays.

Different types of PVA can be used depending on factors including molecular weight, degree of hydrolysis, and other requirements.

Fully hydrolyzed PVA (FHPVA), also known as fully saponified PVA, is a type of PVA where nearly all the acetate groups have been hydrolyzed to form hydroxyl groups. FHPVA typically has a high degree of hydrolysis (above 98%) and exhibits excellent water solubility and film-forming properties. In exemplary embodiments, it is used to improve ink stability, adhesion, and compatibility with aqueous and polar solvents.

Partially hydrolyzed PVA (PHPVA), also known as partially saponified PVA, is a type of PVA where only a portion of the acetate groups have been hydrolyzed to form hydroxyl groups. PHPVA has a lower degree of hydrolysis compared to FHPVA and may exhibit different properties depending on the extent of hydrolysis. In exemplary embodiments, it is used to provide a balance between water solubility, film-forming properties, and mechanical strength.

Low molecular weight PVA, also known as LMW PVA, refers to PVA with a lower average molecular weight compared to standard grades. LMW PVA exhibits improved water solubility, viscosity, and film-forming properties, making it suitable for applications where rapid dissolution and spreading are required. In the exemplary embodiment, it is used to improve ink stability, flow properties, and substrate adhesion.

High molecular weight PVA, also known as HMW PVA, refers to PVA with a higher average molecular weight compared to standard grades. HMW PVA exhibits increased tensile strength, toughness, and film-forming properties, making it suitable for applications where superior mechanical properties are required. In the exemplary embodiment, it is used to improve ink stability, film integrity, and durability on substrates.

Crosslinked PVA, also known as PVA hydrogels or PVA gels, refers to PVA that has been chemically crosslinked to form a three-dimensional network structure. Crosslinked PVA exhibits improved mechanical strength, water retention, and stability compared to uncrosslinked PVA. In the exemplary embodiment, it is used to improve ink stability, rheological properties, and resistance to environmental factors, including humidity and solvent exposure.

Modified PVA refers to PVA derivatives that have been chemically modified to introduce specific functional groups or properties. Modified PVA can exhibit improved properties, including increased water solubility, compatibility with organic solvents, or improved adhesion to substrates. In the exemplary embodiment, modified PVA derivatives are used to improve the binding of biomolecules, including single-stranded DNA (ssDNA), to the surface of graphene, enhancing the performance of graphene-based biosensors. In one exemplary embodiment, Carboxylated PVA (PVA-COOH) is a modified form of PVA where carboxyl (—COOH) functional groups are introduced onto the polymer backbone. These carboxyl groups can facilitate covalent bonding or electrostatic interactions with amine groups present in ssDNA molecules, promoting their immobilization onto the surface of graphene. This modification improves the sensitivity and specificity of graphene-based biosensors for DNA detection. In another exemplary embodiment, laminated PVA (PVA-NH2), a modified form of PVA where amino (—NH2) functional groups are introduced onto the polymer backbone. These amino groups form strong covalent bonds or coordinate with metal ions to anchor ssDNA molecules onto the surface of graphene. Aminated PVA modification improves the stability and efficiency of DNA immobilization, leading to improved sensor performance and detection sensitivity. In another embodiment, grafted PVA, is a PVA derivative where side chains or functional groups are covalently attached to the polymer backbone. These side chains can provide reactive sites for coupling with ssDNA molecules, facilitating their attachment to the surface of graphene through chemical or physical interactions. Grafted PVA modifications offer versatility and tunability in controlling DNA immobilization and sensor performance in graphene-based biosensors. In other embodiments, thiolated PVA, a modified form of PVA where thiol (—SH) functional groups are introduced onto the polymer backbone, is used. Thiol groups can form strong covalent bonds with gold nanoparticles or graphene surfaces, enabling the immobilization of ssDNA molecules through thiol-gold or thiol-graphene interactions. Thiolated PVA modification improves the stability and sensitivity of DNA sensors by facilitating robust immobilization of DNA probes onto the sensor surface.

Other modified polyvinyl alcohol derivatives are used to non-covalently bind biomolecules, including ssDNA, to the surface of graphene biosensors. In one embodiment, Polyvinyl alcohol-graft-polyethyleneimine (PVA-g-PEI), a modified PVA derivative where polyethyleneimine (PEI) chains are grafted onto the PVA backbone, is used to improve the adsorption of ssDNA molecules onto the graphene surface, improving the sensitivity and specificity of graphene-based biosensors for DNA genomic target detection. PEI contains amine groups that can interact with the oxygen-containing functional groups on the surface of graphene through non-covalent interactions, including hydrogen bonding and π-π stacking. In another embodiment, Polyvinyl alcohol-graft-poly (acrylic acid) (PVA-g-PAA), a modified PVA derivative where poly (acrylic acid) (PAA) chains are grafted onto the PVA backbone, is used. PAA contains carboxyl groups that can form hydrogen bonds and electrostatic interactions with ssDNA molecules, facilitating their adsorption onto the graphene surface. This modification improves the immobilization efficiency and stability of DNA probes on the graphene surface, leading to improved sensor performance and genomic target detection sensitivity. In another embodiment, Polyvinyl alcohol-polyethylene glycol (PVA-PEG) copolymers are used to form a hydrophilic coating on the surface of graphene, preventing nonspecific adsorption of proteins and enhancing the selectivity of DNA sensing. Additionally, PEG chains can interact with ssDNA through hydration layers, promoting the non-covalent binding of DNA probes to the graphene surface. PVA-PEG copolymers are synthesized by conjugating polyethylene glycol (PEG) chains to the PVA backbone. PEG is known for its biocompatibility and ability to reduce non-specific adsorption of biomolecules. In another embodiment, Polyvinyl alcohol-polyethyleneimine (PVA-PEI) nanoparticles provide a scaffold for the immobilization of ssDNA molecules through electrostatic interactions. PVA-PEI is synthesized by crosslinking PVA with PEI to form nanostructures. These nanoparticles absorb onto the graphene surface by leveraging the high surface area and biocompatibility of PVA-PEI nanoparticles, enhancing the sensitivity and stability of graphene-based DNA biosensors. In yet another embodiment, Polyvinyl alcohol-functionalized gold nanoparticles (PVA-AuNPs) are used to adsorb onto the graphene surface and provide binding sites for the non-covalent attachment of ssDNA molecules through interactions including electrostatic forces and π-π stacking. The PVA-AuNPs are synthesized by conjugating PVA chains onto the surface of gold nanoparticles. These PVA-AuNPs can improve the immobilization efficiency and stability of DNA probes on the graphene surface, improving the performance of DNA biosensors for sensitive and selective genomic target detection.

Types of Polyethylene Glycol (PEG) are used in a graphene ink based on desired characteristics, particularly its non-toxic nature and biocompatibility with biomolecules and biological samples. Other desirable characteristics of PEG for use in biosensing applications include favorable rheological properties, including solubility, dispersibility, stability; advantages for surface modification; minimization of non-specific binding; hydration layer maintenance, and flexibility in functionalization. For example, PEG's improved solubility and steric hindrance improve ink stability by preventing aggregation of graphene flakes and maintaining their dispersion within the ink. PEG can be used to modify the surface properties of graphene by creating a more hydrophilic and biocompatible interface for binding biomolecules to the surface of graphene. In an exemplary embodiment using covalent binding, PEG is functionalized with amine, and thiol, among other groups, to facilitate better biomolecule attachment and orientation. In another embodiment using non-covalent binding, PEG promotes a more hydrophilic and biocompatible graphene surface, which limits the disruption of the π-π stacking interactions between graphene and the aromatic bases of biomolecules like ssDNA. PEG also reduces biofouling and non-specific adsorption of proteins and other biomolecules.

Low Molecular Weight PEGs (e.g., PEG 200, PEG 400), in the exemplary embodiment, are used to improve the solubility and dispersibility of graphene in various solvents. Their lower molecular weight facilitates better interaction with graphene sheets and can help in preventing restacking or aggregation. Whereas High Molecular Weight PEGs (e.g., PEG 2000, PEG 6000) in other exemplary embodiments are used to create a hydrated layer around the graphene. The hydration layer provides a steric barrier around the graphene flakes, enhancing stability in the dispersion, improving the biocompatibility of the graphene, and minimizing bio-interference.

Functionalized PEGs are chemically modified to include functional groups at one or both ends of their chains. The functional groups interact with molecules or surfaces of other materials through chemical or physical mechanisms. In one embodiment, amine (NH2) groups are used to functionalize PEGs for binding to carboxyl groups or other reactive agents. PEG-Amine groups are used to create a positively charged surface that helps in attracting the phosphate backbone of ssDNA, thereby facilitating better orientation, and spacing of the DNA probes on the surface of the graphene sensor. In another embodiment, thiol (SH) groups are used to functionalize PEGs to form strong bonds with gold and other metals used in nanoparticle-based applications. When used in conjunction with metal nanoparticles or a metal-decorated graphene surface, PEG-Thiol helps to create a more structured and functionalized interface for ssDNA attachment. Similarly, PEG and functionalized PEG, act as blocking agents to cover non-specific active sites on the graphene surface, preventing unwanted interactions and ensuring that ssDNA probes bind specifically where desired. PEG and functionalized PEGs also help maintain a hydrated environment around the graphene sheets, which is crucial for the bioactivity and stability of ssDNA. This hydration layer ensures that ssDNA maintains its native structure and functionality when interacting with target molecules. In yet other embodiments, Carboxyl (COOH) groups are used to functionalize PEGs to react with amine groups, forming amine bonds. In other embodiments, silane is used for surface modification, particularly for glass and metal oxide surfaces, and acrylate allows incorporation into other polymers through free radical reactions.

Biocompatible PEGs serve as a dispersant, binder, and functional additive for improving graphene ink printability, stability, and biocompatibility. In one embodiment, PEG Diacrylate (PEGDA) is used for attaching acrylate groups to the PEG polymer chain ends and can be polymerized using UV light or heat from hydrogels. In another embodiment, PEG Diglycidyl Ether (PEGDGE), which contains reactive epoxy groups that cross-link with other polymers and react with functional groups on the graphene surface, is used to improve mechanical strength and the stability of the printed graphene structures. PEGDGE is also used to create a more hydrophilic and biocompatible graphene surface, which enhances interactions between the graphene surface and deposited biomolecules. In another embodiment, PEG Methacrylate (PEGMA) is used. PEGMA has methacrylate groups that copolymerize with other methacrylate-based monomers, forming networks that provide flexibility and strength to the graphene structures. In another embodiment, PEG Thiol (PEG-SH) is used to form strong bonds with gold or other thiol-reactive surfaces. In another embodiment, PEG-Phospholipid Conjugates are used to improve biocompatibility by mimicking cell membrane components. In another embodiment, PEG-Silane (PEG-Si) is used to improve adhesion and graphene ink stability. PEG-Si contains silane groups that bond to silica, glass, or similar surfaces, improving the adhesion of the ink to the substrate.

PEG Blends are used to improve the biocompatibility, stability, and functionality of graphene surfaces. The PEG blends create an environment conducive to binding biomolecules to graphene surfaces while maintaining high biosensor sensitivity and selectivity. In one embodiment, Branched PEG (B-PEG) is used to provide a dense, stable, and hydrated layer that mimics the natural environment of biomolecules. The B-PEG molecules consist of a central core with multiple PEG chains radiating out and improving the non-covalent binding of probes. In another embodiment, PEG-Biotin is used to bind biotinylated probes to the surface of the graphene. PEG-B molecules have biotin groups attached to the end of the PEG chains. Biotin strongly binds to streptavidin or avidin, which can be used to attach biotinylated probes. This allows for a flexible and reversible binding strategy for probes. In another embodiment, PEG-Poyl (L-lysine) (PEG-PLL) is used to interact with the negatively charged graphene oxide surface, while PEG provides steric stabilization. This interaction improves the binding of negatively charged probes through electrostatic interactions while preventing non-specific adsorption. In another embodiment, PEG-Folic Acid (PEG-FA) is used to improve the specificity of the graphene sensor. PEG conjugated with folic acid targets specific biomolecular interactions, including those with folate receptors.

PEG-ssDNA conjugates are formed by chemically linking the PEG polymer to the ssDNA molecule using specific functional groups. In one embodiment, Thiol-Maleimide chemistries are used. A thiol (—SH) group is introduced at the end of the ssDNA, and the PEG is functionalized with a maleimide group. The thiol group on the ssDNA reacts with the maleimide group on the PEG to form a stable thioether bond. In another embodiment, NHS Ester chemistries are used. An amine (—NH2) group is introduced at the end of the ssDNA, and PEG is functionalized with an NHS ester. The amine group on the ssDNA reacts with the NHS ester on the PEG to form a stable amide bond. In another embodiment, Click Chemistries (Azide-Alkyne Cycloaddition) are used. An azide (—NH4) group is introduced on the ssDNA, and PEG is functionalized with an alkyne group. The azide and alkyne groups react in the presence of a copper catalyst to form a triazole linkage.

The types of Polyurethane (PU) used in graphene ink are based on desired characteristics. Different types of PU can be used depending on factors including flexibility, elasticity, mechanical strength, adhesion, film formation, chemical resistance, and high cross-link density. Polyurethane (PU) is used as a binder and film-forming agent to improve adhesion, which ensures that the printed layer remains intact and durable. PU is also used to improve flexibility and elasticity. It also promotes chemical resistance, limiting degradation from environmental factors, including moisture, and resulting in greater reliability of printed structures over time. PU also improves dispersibility and stability by preventing graphene flake aggregation and sedimentation.

In one embodiment, Thermoplastic Polyurethane (TPU) is used to improve the mechanical strength by providing a flexible, yet durable matrix that supports graphene flakes. TPU's elasticity helps in absorbing mechanical stress and strains, preventing cracks, and maintaining the integrity of the graphene film. TPU also provides a protective barrier, shielding graphene flakes from environmental factors including moisture, chemicals, and UV radiation. The hydrophobic nature of TPU facilitates hydrophobic-hydrophobic interactions between the graphene surface and deposited biomolecules, aiding in the stable non-covalent binding of ssDNA. TPU also facilitates non-covalent interactions like π-π stacking and hydrogen bonding between the nucleobases of ssDNA and the graphene surface. TPU is also highly compatible with aqueous environments, making it easier to work with various buffers required for ssDNA binding and hybridization without compromising the integrity of the printed film. TPU also helps achieve the optimal viscosity and thixotropic behavior required for various printing methods.

In another embodiment, a non-toxic waterborne polyurethane (WPU) is used to improve film-forming, adhesion, and mechanical strength and provide chemical resistance. WPU, due to its low toxicity, facilitates the binding of biomolecules to graphene surfaces. WPU improves both hydrophilic and hydrophobic interactions between ssDNA and the graphene surface, providing greater binding stability. These interactions create an environment that supports hydrogen bonding and π-π stacking interactions between the nucleobases of ssDNA and the graphene surface.

In another embodiment, polyurethane acrylate (PUA) is used for rapid curing using ultraviolet light or UV curing. Rapid UV curing allows for high-volume production while providing improved mechanical strength, durability, and improved adhesion.

In yet another embodiment, thermosetting polyurethane (TSU) is used to form a highly cross-linked polymer network, providing superior mechanical strength and chemical resistance. The cross-linked structure offers superior dimensional stability and structural integrity. The strong structural support forms a robust and stable matrix that encapsulates the graphene flakes, ensuring that the surface remains flat and well-aligned, which is essential for binding biomolecules to the surface of graphene-based biosensors. Like other forms of polyurethane, TSU provides improved dispersion and adhesion. TSU achieves a uniform dispersion of graphene, preventing aggregation and ensuring a high surface area for binding biomolecule binding. TSU also provides excellent adhesion to a wide variety of substrates, which maintains the integrity of the printed graphene layer.

In another embodiment, polyurethane elastomers (PUE) are used to promote hydrophobic interactions between hydrophobic regions of the ssDNA and the graphene surface. This interaction further stabilizes the binding of biomolecules to the graphene surface. The PUE matrix supports hydrogen bonding and π-π stacking interactions between the ssDNA and the graphene surface. These non-covalent interactions are for stable and specific ssDNA binding. PUE has the advantage of providing a barrier against moisture and environmental factors that could degrade the graphene, ensuring the long-term stability and reliability of the graphene biosensor.

The types of Polystyrene (PS) used in graphene ink are based on desired characteristics. Different types of PS can be used depending on factors including dispersibility, stability, film formation, mechanical strength, adhesion, chemical and environmental resistance, and electrical properties. Polystyrene achieves uniform dispersion of graphene flakes. This ensures a consistent surface area for binding biomolecules to the surface of the graphene, which is critical for reliable sensor performance. The uniform dispersion also provides a stable matrix that prevents aggregation of graphene flakes, maintaining the functional properties of the ink over time. The uniform dispersion and stable matrix produce smooth and uniform films, which are essential for creating a reproducible response from biosensors. Polystyrene's hydrophobic nature can improve hydrophobic-hydrophobic interactions between the ssDNA and the graphene surface. These interactions are for stable non-covalent binding. The aromatic structure of polystyrene can facilitate π-π stacking interactions and van der Waals forces between the nucleobases of ssDNA and the graphene, further stabilizing the binding of biomolecules. PS increases the effective surface area available for ssDNA binding by preventing graphene aggregation and promoting a more open structure. This ensures a higher density of binding sites for ssDNA. PS also imparts flexibility and toughness to the printed films, allowing them to withstand mechanical stress and deformation. This is particularly important for flexible and wearable biosensors.

In one embodiment, general-purpose polystyrene (GPPS) improves graphene inks by providing excellent film formation, mechanical strength, uniform dispersion, chemical compatibility, and facilitating functionalization for the non-covalent binding of biomolecules. In another embodiment, High Impact Polystyrene (HIPS) is used for increased durability and toughness. HIPS is known for its high impact resistance, which is particularly useful where printed graphene films are subject to mechanical stress or handling. The addition of rubber particles to HIPS provides flexibility to the printed graphene films. This flexibility ensures that the films can bend and stretch without cracking, making HIPS-graphene inks suitable for flexible electronics and wearable devices. HIPS helps achieve a uniform dispersion of graphene flakes within the ink. The rubber particles in HIPS can prevent graphene from aggregating, ensuring a consistent distribution of graphene throughout the ink. The presence of HIPS provides a stable matrix that supports the graphene flakes, preventing them from settling or clumping over time. HIPS provides excellent adhesion to a variety of substrates, including plastics, glass, and flexible polymers. This strong adhesion ensures that the printed graphene films remain securely attached to the substrate, which is critical for maintaining the functionality of the printed electronics or sensors. The mechanical properties of HIPS allow for better control over the thickness of the printed films, which can be important for optimizing the electrical properties of graphene ink.

In another embodiment, Polystyrene-Butadiene-Styrene (SBS) is used to improve film flexibility and elasticity. SBS is a block copolymer consisting of styrene and butadiene that ensures that the film can endure repeated mechanical stress without cracking or losing functionality. This improved mechanical strength is optimal for applications that include wearables or implantables that demand a high level of flexibility and elasticity of the printed films.

In another embodiment, polystyrene sulfonate (PSS) is used to improve conductivity. PSS, when used in combination with conductive polymers like polythiophene derivatives (e.g., poly (4,4-ethylenedioxythiophene) or PEDOT), can significantly improve the conductivity of the composite material. PEDOT: PSS is a combination where PSS acts as a dopant and stabilizer for the conductive PEDOT. PSS also improves the conductivity of graphene by acting as a dopant. The negative charges on PSS can interact with graphene, modifying its electronic properties and improving its conductivity. The sulfonate groups on PSS provide functional sites for further chemical modifications or for binding biomolecules. This is particularly useful in biosensors where PSS can facilitate the attachment of biomolecules like ssDNA to the graphene surface through non-covalent interactions. PSS is hydrophilic, which makes it easier to functionalize the graphene surface with hydrophilic molecules or biomolecules. This improves the compatibility of the graphene ink with aqueous solutions and biological environments.

The types of Acrylic Polymers used in exemplary graphene ink formulations. Acrylic polymers provide a medium that enables the stable dispersion of graphene particles and enhances the overall performance of the ink. Acrylic polymers help stabilize graphene particles in the ink, preventing agglomeration and sedimentation. This ensures a uniform dispersion of graphene, which is critical for consistent performance. Acrylic polymers can interact with the surface of graphene sheets, providing steric and electrostatic stabilization. This interaction helps keep the graphene sheets well-dispersed in the liquid medium. Acrylic polymers can adjust the viscosity of graphene inks to suit different printing techniques, including screen printing, inkjet printing, aerosol printing, pad printing, flexographic printing, and other methods. Proper viscosity is essential for ensuring smooth application and preventing clogging in printing equipment. These polymers can impart thixotropic properties to the ink, making it more viscous under low-shear conditions (at rest) and less viscous under high-shear conditions (during printing). This behavior is beneficial for achieving precise and clean prints. Acrylic polymers act as a binding agent, helping to form a cohesive film of graphene on the substrate once the ink is applied and dried or cured. This ensures good adhesion of the graphene layer to the substrate, which is vital for the durability and functionality of the printed structures. Acrylic polymers are compatible with a wide range of solvents, which allows for the formulation of graphene inks that can be tailored to different printing processes and substrate materials. The choice of solvent can influence the drying time, adhesion, and overall performance of the ink. While ensuring dispersion stability and film formation, acrylic polymers are formulated to minimize any adverse effects on the electrical conductivity of the graphene. Proper formulation techniques ensure that the graphene retains its excellent conductive properties in the final printed product. An exemplary embodiment is Poly (methyl methacrylate) (PMMA), which has excellent film-forming properties, mechanical stability, and transparency. PMMA can improve the dispersion and stabilization of graphene within the ink matrix. Another exemplary embodiment is Styrene-acrylic emulsions. These polymers offer good dispersion stability and are known for improving the water and oxygen resistance of the resulting graphene inks. They are particularly effective when combined with aqueous graphene dispersions. Another exemplary embodiment is Acrylic-based UV-curable formulations. These inks utilize acrylic polymers that can be cured using UV light, facilitating the reduction of graphene oxide to graphene during the curing process. This method provides a straightforward and efficient way to produce conductive patterns.

The types of Epoxy resin used in exemplary graphene ink formulas. Epoxy resins are used in graphene inks primarily for their excellent adhesion, mechanical strength, and chemical resistance properties. Epoxy resins help in stabilizing graphene dispersions. Graphene nanoparticles can be effectively dispersed in epoxy resin matrices, which prevents agglomeration and ensures uniform distribution throughout the ink. This results in better conductive properties and a more consistent performance of the printed materials. When cured, epoxy resins provide strong mechanical properties and flexibility to the printed graphene layers. This is for applications that require durable and flexible conductive patterns, including flexible electronics and wearable devices. Epoxy resins offer excellent thermal and chemical resistance, which protects the graphene structures in harsh environments. This makes graphene inks suitable for a wider range of industrial applications where exposure to chemicals and high temperatures might be a concern. The curing process of epoxy resins can also aid in the reduction of graphene oxide to graphene, enhancing the electrical conductivity of the printed inks. This is particularly useful in applications requiring high electrical performance, including printed circuits and sensors. Epoxy resin-based graphene inks can be used in various printing techniques, including screen printing, inkjet printing, flexographic printing, and other methods. The versatility of application methods makes them suitable for a broad range of industrial and commercial uses. An exemplary embodiment is Bisphenol A Epoxy Resin, used for its good mechanical properties and adhesion, it is commonly used due to its low viscosity and ease of processing. Another exemplary embodiment is Cycloaliphatic Epoxy Resin, which offers high-temperature and UV resistance, making it suitable for applications requiring durability under extreme conditions. Another exemplary embodiment is the Diglycidyl Ester of Hexahydrophthalic Acid, which provides excellent thermal stability and corrosion resistance. Another exemplary embodiment is Tri-functional and Tetra-functional Epoxy Resins. These resins offer improved mechanical properties and are used for applications for higher structural integrity.

The types of Polyimides used in exemplary graphene ink formulas. Polyimides are used in graphene inks primarily for their excellent thermal stability, chemical resistance, and mechanical properties. Polyimides can act as a robust matrix for dispersing graphene, enhancing the uniformity and stability of the ink. Their thermal stability makes them ideal for applications that involve high temperatures. Polyimides provide resistance to solvents and chemicals, protecting the graphene structures in harsh environments. They improve the mechanical properties of the printed films, ensuring durability and flexibility. One exemplary embodiment is Kapton, a polyimide known for its exceptional thermal stability and mechanical strength. Another exemplary embodiment is Poly(o-phenylenediamine) (PoPD). This polyimide is used to create noncovalent functionalized graphene composites. The interaction between PoPD and graphene improves the thermal and mechanical properties of the ink, making it suitable for applications requiring wear resistance and high-temperature performance. Another exemplary embodiment is Polyamic Acid-based Polyimides. These are synthesized through a two-step process and are utilized for their ability to form flexible and strong films. They provide excellent adhesion and stability, which are critical for high-performance printed electronics and sensors. Another exemplary embodiment is Amine-functionalized Polyimides. These polyimides are modified to improve compatibility with graphene oxide. The functionalization helps in better dispersion of graphene within the polyimide matrix, enhancing the mechanical and dielectric properties of the resulting composite films. Another exemplary embodiment is Fluorinated Polyimides. These polyimides are used for their excellent chemical resistance and stability under extreme conditions. They help improve the adhesion of graphene layers and are often used in applications that demand high reliability and durability.

Layering and loading of molecules onto the surface of Van der Waal (VDW) material, including graphene, for increasing electrical resistance. The electrical resistance of the VDW material includes graphene changes when molecules are deposited on the surface through at least one of several mechanisms, including π-π stacking, Van der Waal forces, electrostatic interactions, hydrophobic interactions, charge transfer, chemical doping, physisorption, and hydrogen bonding.

π-π stacking occurs when aromatic molecules or rings, including polycyclic aromatic hydrocarbons, organic dyes, or DNA bases, are deposited on a VDW material, including graphene, and their r-electron systems interact with the r-electrons in graphene's sp²-bonded carbon lattice, causing a disruption of the delocalized r-electron system of the VDW material, including graphene. This disruption lowers the electrical conductivity and introduces scattering sites for electrons, thus increasing the electrical resistance of the VDW material, such as a graphene sensor. The r-r interaction can also modify the local electronic environment in the VDW material, including graphene, creating localized states that can trap charge carriers, which reduces the number of free charge carriers available for conduction and thereby increasing electrical resistance.

Van der Waal forces are interactions that occur between all atoms and molecules due to the temporary dipoles forming as electrons move and as molecules adsorb onto the surface of a Van der Waal material, including graphene. These interactions act as physical barriers impeding the flow of electrons across the surface of the VDW material, creating a scattering effect, and resulting in an increase in the electrical resistance of the Van der Waal material sensor. Van der Waal forces can also change the local dielectric environment through dielectric screening, which occurs when molecules adsorb on the surface of the VDW material, including graphene. The dielectric screening alters the electric field distribution and can reduce the mobility of charge carriers, leading to an increase in the electrical resistance of the Van der Waal material sensor.

Electrostatic interactions occur between charged and polar molecules and Van der Waal materials, including graphene, through charge distribution, leading to positive or negative charge of the Van der Waal material, including graphene, facilitating interactions with charged or polar groups on molecules, leading to increased electron scattering and higher electrical resistance of the van der Waal material sensor. The increased electron scattering leads to a reduction in the mobility of the charge carriers, which directly translates into increased electrical resistance. This is because the mean free path of the carriers is shortened, meaning they are more frequently deflected off course by the electrostatic fields created by the adsorbed molecules.

Hydrophobic interactions occur when nonpolar molecules or molecular regions encounter each other in an aqueous environment. Van der Waal materials, including graphene, being largely hydrophobic and often nonpolar, can interact with the hydrophobic regions of the molecules when molecules absorb on the surface of the van der Waal material, including graphene, acting as barriers to the electron flow, increasing electron scattering, and increasing the electrical resistance of the van der Waal material sensor. Hydrophobic interactions can modify the surface energy of graphene, potentially leading to changes in the electronic properties, including the density of states at the Fermi level. These changes can impact the availability of charge carriers and their mobility, further contributing to an increase in electrical resistance.

Charge transfer occurs when electrons are transferred from a donor molecule or region to an acceptor molecule or region. This occurs when molecules adsorb onto the van der Waal material, including graphene, altering the carrier concentration of the van der Waal material, including graphene. Charge transfer through either donating or accepting electrons modifies the charge density and directly affects electrical resistance, increasing the electrical resistance of the van der Waal material sensor by scattering charge carriers or inducing defects in the van der Waal material, including graphene, and reducing the charge carriers. Any minor charge transfer or alteration in local electronic density can shift the Fermi level of graphene, potentially moving it closer to the Dirac point, where graphene has minimal conductivity. This shift can lead to an increase in electrical resistance. Depending on the electronic properties of the adsorbed molecule, there can be a transfer of charge between the molecule and graphene. If the molecule donates electrons, the graphene can become more n-type (increasing electron density), potentially decreasing resistance. Conversely, if the molecule accepts electrons, it can make graphene more p-type (increasing hole density), potentially increasing resistance.

Chemical doping occurs when molecules modify the electronic properties of van der Waal material, including graphene, without forming strong covalent bonds. Chemical doping directly affects electrical resistance depending on the nature of the dopant (N-type—molecules that donate electrons or P-type—molecules that withdraw electrons). The electrical resistance of the van der Waal material sensor increased by introducing defects and scattering the charge carriers. Mechanisms of chemical doping can also include π-π stacking, charge transfer, electrostatic interactions, modification of band structure, and other forms of van der Waal forces. Chemical doping can induce changes in the band structure of graphene, including opening a bandgap or creating localized states. These modifications can affect how charge carriers move through the material. The introduction of a bandgap or localized states can trap charge carriers or increase the energy required for conduction, thereby increasing the electrical resistance.

Atoms, ions, and molecules combine to form new hybrid orbitals with van der Waal materials, including graphene. When atoms, ions, or molecules interact with van der Waal materials, including graphene, to form new hybrid orbitals, the process involves the overlap of molecular orbitals between the adsorbed atoms, ions, and molecules and the graphene surface. Orbital overlap is facilitated by energy matching, charge transfer, and functionalization of the van der Waal material, including graphene. These interactions lead to the formation of hybrid orbitals that have characteristics of both the van der Waal material, including graphene, and the adsorbed atom, ion, or molecule.

Orbital overlap occurs when atoms, ions, or molecules absorb on the surface of van der Waal materials, including graphene. The π orbitals of the van der Waal material include graphene overlap with the molecular orbitals of the absorbed atoms, ions, or molecules. The overlap is significant when the absorbed atom, ion, or molecule has π orbitals, including aromatic compounds. The interaction between the π orbitals of the atom, ion, or molecule and the delocalized π orbitals of the van der Waal material, including graphene, led to a new set of hybrid orbitals.

Energy matching occurs when the energy levels of the interacting orbitals of the van der Waal material, including graphene and the absorbed atom, ion, or molecule, are relatively close, enabling orbital overlap. Energy matching results in stronger interactions, more effective hybridization, and the formation of new hybrid orbitals. The new hybrid orbitals have characteristics that are intermediated between the contributing orbitals and are lower in energy, resulting in a more stable molecular configuration.

Charge transfer occurs when atoms, ions, and molecules adsorb onto van der Waal materials, including graphene. Electrons are transferred from at least one atom, ion, or molecule (the donor) to another (the acceptor). Depending on the nature of the adsorbed atom, ion, or molecule, there may be a transfer of charge between the adsorbed atom, ion, or molecule and the van der Waal material, including graphene. This charge transfer alters the electronic structure of both the adsorbed atom, ion, or molecule and the van der Waal material, including graphene, leading to the formation of new hybrid orbitals. For example, if the adsorbed atom, ion, or molecule is an electron donor or acceptor, it can transfer electrons to the van der Waal material, including graphene, or transfer them away from the van der Waal material, including graphene.

New electronic states occur when atoms, ions, and molecules adsorb onto the surface of the van der Waal material, including graphene. The interaction between the adsorbed atom, ion, and molecule and the van der Waal material including graphene creates new hybrid electronic states that are combinations of the original states of the absorbed atoms, ions, and molecules and the van der Waal material includes graphene. These hybrid states can have different properties compared to the original states, including changes in the density of states, band structure, and electron conductivity.

Functionalization occurs when atoms, ions, or molecules adsorb onto the van der Waal materials, including graphene. Functionalization improves specific interactions between the absorbed atoms, ions, and molecules by creating localized sites that favor hybridization with certain types of atoms, ions, and molecules. The specific interactions modify the electronic properties of the van der Waal material, including graphene, and lead to the formation of new hybrid orbitals.

Sp2 hybridization and exemplary methods for binding molecules to van der Waal materials include graphene. sp2 hybridization of van der Waal materials, including graphene, enables the binding of molecules to van der Waal materials, including graphene, by forming new hybrid orbitals. sp2 hybridization is a type of orbital hybridization in chemistry where one “S” orbital and two “P” orbitals from the same atom combine to form three equivalent hybrid orbitals. This type of hybridization forms double bonds and planar structures, 120 degrees apart from each other, leading to a trigonal planar geometry, where a central atom is bonded to three other atoms positioned at the corners of an equilateral triangle. Each sp2 hybrid orbital can overlap with another atomic orbital to form sigma (a) bonds, which are strong and lie along the axis connecting the bonded atoms. The unhybridized “P” orbital (PZ) perpendicular to the plane of the sp2 hybrid orbitals can overlap sideways with another “P” orbital to form a pi (π) bond. This type of bond is found in double bonds, alongside a sigma (σ) bond. The 120-degree bond angles and planar structure associated with sp2 hybridization are in determining the physical and chemical properties of molecules, including reactivity and interaction with other molecules. The presence of pi (π) bonds in sp2 hybridized molecules influences the electronic properties, including conductivity, and chemical reactivity, particularly in conjugated systems.

Interactions occur when van der Waal materials, including graphene's sp2 hybridized carbon atoms, form a hexagonal lattice with delocalized r-electrons above and below the plane of the sheet. These r-electrons interact with the r-electrons of other aromatic molecules, including benzene rings, leading to strong r-r stacking interactions. This type of interaction occurs with molecules that have aromatic systems, including benzene rings, polycyclic aromatic hydrocarbons, DNA bases, and organic dyes.

Van der Waal forces between a van der Waal material, including graphene, and an adsorbed molecule on the graphene surface are enabled by the van der Waal material's large surface area. Van der Waal forces are non-covalent interactions that arise from temporary dipoles induced in molecular atoms. London dispersion forces result from temporary fluctuations in the electron distribution within atoms or molecules, creating transient dipoles that induce corresponding dipoles in nearby molecules. Dipole-dipole interactions occur between molecules that have permanent dipoles. Dipole-induced dipole interactions occur when a polar molecule induces a dipole in a nonpolar molecule. London dispersion forces are facilitated by surface modification techniques, optimization of surface morphology, and utilization of layered hybrid materials.

Electrostatic interactions occur from van der Waal materials, including graphene, having regions of positive and negative charge due to the distribution of its π-electron cloud. Such charge distribution is further improved by introducing defects, impurities, or functionalization. Molecules with polar groups or charged species interact electrostatically with these regions, enhancing the adsorption of molecules onto the surface of van der Waal materials, including graphene. Hydrophobic interactions occur between more than one hydrophobic molecule. These hydrophobic, nonpolar molecules associate with each other to avoid contact with water. Van der Waal materials, including graphene, are hydrophobic, enabling the adsorption of other hydrophobic molecules by hydrophobic interactions.

The graphene production processes disclosed herein enable scalable synthesis of graphene nanoflakes with controlled lateral dimensions, layer number, and surface chemistry. Production routes such as detonation, plasma synthesis, liquid exfoliation, and mechanical exfoliation are configured to generate graphene materials exhibiting tailored defect densities and oxygen-containing functional groups that facilitate downstream functionalization and dispersion.

Detonation-based synthesis is performed by initiating controlled high-energy decomposition of carbon precursors, producing few-layer graphene particulates. Subsequent purification steps remove amorphous carbon and metal residues while staged cooling suppresses agglomeration. This combination of high-energy generation and controlled recovery yields highly dispersible graphene particulates suitable for further modification.

Plasma-based production introduces carbon-containing gases into an energized plasma chamber under controlled temperature and pressure conditions, enabling fragmentation and recombination of carbon species to form graphene. In-situ plasma purification mitigates amorphous carbon accumulation, producing graphene with defined surface functionality and enhanced electronic uniformity.

Following production, graphene purification is conducted to remove residual metals, solvents, amorphous carbon, or unexfoliated graphite. Purification may include solvent washing, filtration, staged centrifugation, thermal annealing, or plasma-assisted cleanup. These processes reduce unwanted species on the graphene surface and improve consistency of layer thickness, conductivity, and chemical reactivity.

In various embodiments, purification includes sequential centrifugation to isolate monolayer, bilayer, and few-layer fractions according to sedimentation behavior. This enables production of narrowly distributed graphene materials with heightened uniformity for inks, coatings, and composite applications.

A graphene drying process is performed after purification to remove retained solvents, moisture, and adsorbed species from the graphene surfaces. Drying may be performed using vacuum drying, thermal annealing, or inert-gas flow to ensure that functional groups remain intact while eliminating residual liquids that may interfere with functionalization or dispersion.

In select embodiments, drying is combined with controlled heating to remove oxygenated impurities without degrading intrinsic graphene structure. Vacuum-assisted drying preserves dispersion-ready material and enhances reproducibility across production batches.

In certain embodiments, purified and dried graphene layers are transferred using a wet-transfer or substrate-assisted process. Transfer steps relocate exfoliated graphene onto target substrates such as polymers, metals, or oxides, enabling integration into electronic, sensing, barrier, or coating platforms.

Transfer operations may employ water-soluble carrier layers, solvent-controlled adhesion tuning, or substrate heating to promote uniform placement while preserving structural integrity of the graphene film.

Graphene functionalization modifies surface chemistry to enhance compatibility with solvents, polymers, biomolecules, or composite matrices. Functionalization approaches include covalent attachment, non-covalent adsorption, silanization, POSS grafting, plasma activation, or chemical-phase treatment. These techniques introduce controlled functional groups that adjust reactivity and dispersion properties.

Covalent modification is performed by creating reactive sites through oxidation, plasma exposure, or electrochemical activation, followed by attachment of functional groups such as acyl, diazonium-derived, ester, amine, or carboxyl moieties. Non-covalent approaches employ π-π stacking, hydrophobic interactions, or van der Waals forces to adsorb functional molecules without disrupting graphene's sp² structure.

Silanization and POSS-based methods introduce molecular bridges or inorganic-organic cages to provide steric stabilization and reactive termini. Plasma-based routes incorporate oxygen- or nitrogen-containing groups via ionized gas treatment, enhancing wettability and adhesion.

Graphene dispersion processes incorporate functionalized graphene into solvent systems including organic solvents, non-organic solvents, surfactants, POSS derivatives, silane coupling agents, and aqueous media. Dispersion is conducted via ultrasonication, high-shear mixing, or temperature-controlled processing to produce stable suspensions with controlled zeta potential and particle size uniformity.

Surfactant-assisted dispersion uses anionic, cationic, non-ionic, or zwitterionic surfactants to provide steric and electrostatic stabilization around graphene nanoflakes. POSS derivatives act as steric modifiers that enhance long-term colloidal stability, while silane systems create chemical bridges between graphene surfaces and solvent environments.

Aqueous and hybrid aqueous-organic dispersion systems enable environmentally friendly and biocompatible formulations. Buffer systems, biopolymers, and controlled solvent mixtures maintain dispersion quality across temperature, pH, or storage variations. These dispersion approaches support downstream use in printing, coating, sensing, and composite manufacturing.

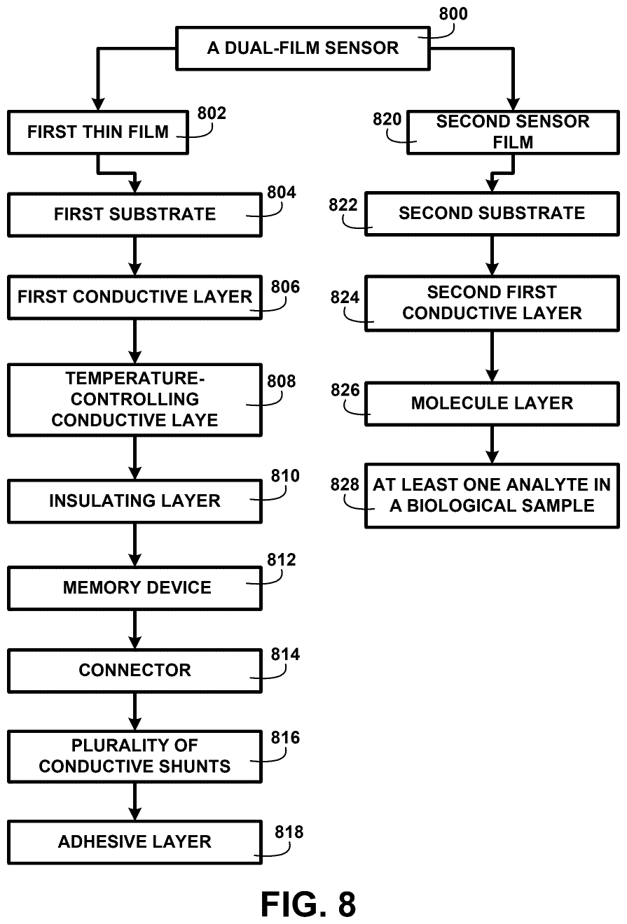

FIG. 8 shows a block diagram of a dual-film sensor of one embodiment. FIG. 8 shows an apparatus including a dual-film sensor comprising a first thin film coupled to a first substrate. A first conductive layer is deposited on the first substrate. A temperature-controlling conductive layer deposited on at least a portion of the first conductive layer is configured to heat a biological sample to facilitate cellular lysis. An insulating layer is deposited over at least the temperature-controlling conductive layer and a portion of the first conductive layer. A memory device electrically coupled to the first conductive layer and configured to store analyte signature data or protocol instructions. A connector configured for the first conductive layer to interface with an external processing or reader device. A plurality of conductive shunts extending from the first conductive layer and having an adhesive layer disposed over the plurality of conductive shunts extending from the first conductive layer of the first thin film and configured to mechanically and electrically bond the first thin film to a second sensor film.

A second sensor film is coupled to a second substrate. A second conductive layer deposited over the second first conductive layer to form an electrode pattern, wherein the. A second first conductive layer is deposited over the second first conductive layer. A molecule layer disposed on the second conductive layer and configured to selectively bind to at least one analyte in a biological sample. The conductive shunts electrically couple the first thin film to the second sensor film to enable signal and power transmission between dual-film layers.

The foregoing has described the principles, embodiments, and modes of operation of the present invention. However, the invention should not be construed as being limited to the embodiments discussed. The above-described embodiments should be regarded as illustrative rather than restrictive, and it should be appreciated that variations may be made in those embodiments by workers skilled in the art without departing from the scope of the present invention as defined by the following claims.

What is claimed is:

1. A method for producing a functionalized graphene material, comprising:
creating graphene powder via at least one exfoliation or oxidation technique;
introducing oxygen-containing functional groups to the graphene powder using a functionalization process to create graphene oxide from the graphene powder;
receiving carbon material and oxidizing agents in a reactor vessel coupled to component containers configured to deliver predetermined quantities of the carbon material and oxidizing agents to obtain a uniform liquid reaction mixture;
controlling the uniform liquid reaction by continuously mixing the carbon material and oxidizing agents, and further using a reflux condenser to manage exothermic reactions;
a cooling system coupled to the reactor vessel to regulate temperature during addition of oxidizing agents to reduce agglomeration of the graphene powder into solid grapheme clusters;
separating solid graphene clusters from the liquid reaction mixture using a filtration system with filter paper or membranes, depending on the desired particle size of the graphene oxide powder; and
separating the graphene oxide from the liquid using a centrifuge coupled to the filtration system configured to wash and purify the graphene oxide, and monitoring and adjusting pH levels to a predetermined pH level during washing.

2. The method for producing a functionalized graphene material of claim 1, further comprising characterizing the graphene powder by Raman spectroscopy.

3. The method for producing a functionalized graphene material of claim 1, further comprising a pH-adjustment subsystem configured to introduce an acid, base, or buffer to achieve a predetermined target pH level to regulate amphoteric surfactants acidic and basic groups and can act as either anionic or cationic surfactants depending on the pH of the solution.

4. The method for producing a functionalized graphene material of claim 1, wherein the cooling system maintains the reaction temperature within a predetermined temperature range.

5. The method for producing a functionalized graphene material of claim 1, further comprising dispersing the functionalized graphene into a hydrophobic fluid.

6. The method for producing a functionalized graphene material of claim 5, wherein the hydrophobic fluid comprises at least one alcohol, terpene, or glycol.

7. The method for producing a functionalized graphene material of claim 5, further comprising combining the functionalized graphene with a polymer binder dispersed in the hydrophobic fluid.

8. A method for producing and purifying a functionalized graphene material, comprising:
creating graphene powder via at least one exfoliation or oxidation technique;
introducing oxygen-containing functional groups to the graphene powder using a functionalization process to create graphene oxide from the graphene powder;
receiving carbon material and oxidizing agents in a reactor vessel coupled to component containers configured to deliver predetermined quantities of the carbon material and oxidizing agents to obtain a uniform liquid reaction;
controlling the uniform liquid reaction by continuously mixing the carbon material and oxidizing agents, and further using a reflux condenser to manage exothermic reactions;
a cooling system coupled to the reactor vessel to regulate temperature during addition of oxidizing agents to reduce agglomeration of the graphene powder into solid grapheme clusters;
separating solid graphene clusters from the liquid reaction mixture using a filtration system with filter paper or membranes, depending on the desired particle size of the graphene oxide powder;
separating the graphene oxide from the liquid using a centrifuge coupled to the filtration system configured to wash and purify the graphene oxide, and monitoring and adjusting pH levels to a predetermined pH level during washing; and
drying the graphene oxide at low temperatures to avoid decomposition using drying equipment.

9. The method for producing and purifying a functionalized graphene material of claim 8, further comprising a pH-adjustment subsystem configured to introduce an acid, base, or buffer to achieve a predetermined target pH level to regulate amphoteric surfactants acidic and basic groups and can act as either anionic or cationic surfactants depending on the pH of the solution.

10. The method for producing and purifying a functionalized graphene material of claim 8, further comprising drying the purified graphene oxide in a vacuum oven.

11. The method for producing and purifying a functionalized graphene material of claim 8, further comprising storing the dried graphene oxide in a desiccator.

12. The method for producing and purifying a functionalized graphene material of claim 8, wherein the filtration system comprises filter paper or membranes selected to control graphene-oxide particle size.

13. The method for producing and purifying a functionalized graphene material of claim 8, wherein the centrifuge performs multiple sequential wash cycles.

14. The method for producing and purifying a functionalized graphene material of claim 8, further comprising analyzing the purified graphene oxide using characterization spectroscopy.

15. A method for producing, purifying, and formulating a functionalized graphene ink, comprising:
creating graphene powder via at least one exfoliation or oxidation technique;
exposing the graphene powder to a functionalization process to introduce oxygen-containing functional groups;
receiving carbon material and oxidizing agents in a reactor vessel coupled to component containers configured to deliver predetermined quantities of the carbon material and oxidizing agents to obtain a uniform liquid reaction mixture;
controlling the uniform liquid reaction by continuously mixing the carbon material and oxidizing agents, and further using a reflux condenser to manage exothermic reactions;
a cooling system coupled to the reactor vessel to regulate temperature during addition of oxidizing agents to reduce agglomeration of the graphene powder into solid grapheme clusters;
comparing the size of the oxidized graphene to match an order specification using an oxidized graphene characterization detector to determine the size of the oxidized graphene;
separating solid graphene clusters from the liquid reaction mixture using a filtration system with filter paper or membranes, depending on the desired particle size of the graphene oxide powder;
separating the graphene oxide from the liquid using a centrifuge coupled to the filtration system configured to wash and purify the graphene oxide, and monitoring and adjusting pH levels to a predetermined pH level during washing; and
drying the graphene oxide at low temperatures to avoid decomposition using drying equipment;
dispersing the functionalized graphene into a hydrophobic fluid; and
combining predetermined quantities of oxidized graphene, hydrophobic fluid, polymer binder, and additives in an ink mixer configured to produce a functionalized graphene ink formulation.

16. The method for producing, purifying, and formulating a functionalized graphene ink of claim 15, wherein the ink mixer combines materials at a predetermined mixing ratio.

17. The method for producing, purifying, and formulating a functionalized graphene ink of claim 15, further comprising depositing the graphene ink onto a substrate in a predetermined pattern.

18. The method for producing, purifying, and formulating a functionalized graphene ink of claim 15, wherein the polymer binder comprises at least one of PVP, PVA, PEG, polyurethane, or acrylic polymer.

19. The method for producing, purifying, and formulating a functionalized graphene ink of claim 15, wherein the additives comprise at least one of dispersing agents, wetting agents, rheology modifiers, plasticizers, antifoaming agents, UV stabilizers, or adhesion promoters.

20. The method for producing, purifying, and formulating a functionalized graphene ink of claim 15, further comprising drying the deposited graphene ink under vacuum to form a printed graphene structure.
